(19) United States
(12) Patent Application Publication
(10) Pub. No.: US 2024/0093288 A1
(43) Pub. Date: Mar. 21, 2024

(54) Ribosomal profiling in single cells

(71) Applicant: Koninklijke Nederlandse Akademie van Wetenschappen, Amsterdam (NL)

(72) Inventors: Alexander van Oudenaarden, Utrecht (NL); Michael Vaninsberghe, Utrecht (NL)

(73) Assignee: Koninklijke Nederlandse Akademie van Wetenschappen, Amsterdam (NL)

(21) Appl. No.: 18/254,179

(22) PCT Filed: Nov. 25, 2021
(86) PCT No.: PCT/EP2021/082952

§ 371 Date: May 24, 2023

(30) Foreign Application Priority Data
Nov. 25, 2020 (EP) 20209743.2

(51) Int. Cl. C12Q 1/6869; G16B 5/00; G16B 35/00

(52) U.S. Cl. CPC C12Q 1/6869; G16B 5/00; G16B 35/00

(57) ABSTRACT

The invention pertains to method for ribosome profiling at a single cell resolution. The method comprises the steps of i) lysing a single cell; ii) digesting the RNA with a ribonuclease, thereby generating an ribosome footprint containing RNA molecules that are protected against digestion; iii) Inactivating the ribonuclease and releasing the RNA molecules from the ribosomes; iv) end repairing the released RNA; v) constructing an RNA library from the end-repaired RNA molecules; vi) size selecting part of the prepared RNA library for fragments having an insert size of about 20-40 nucleotides; vii) sequencing the size selected RNA library; and viii) determining the translatome of the single cell.

### FIELD OF THE INVENTION

The present invention relates to the field of genetic profiling. More in particular, the invention is in the field of transcriptomics and translatomics. The invention concerns a method for ribosome profiling at a single cell resolution.

### BACKGROUND

In recent years novel single-cell sequencing methods have allowed an in-depth analysis of the diversity of cell types and cell states in a wide range of organisms. These novel tools predominantly focus on sequencing the genomes (see e.g. Navin, N. et al. Tumour evolution inferred by single-cell sequencing (2011), Nature, 472, 90-94), epigenomes (see e.g. Smallwood, S. A. et al. Single-cell genome-wide bisulfite sequencing for assessing epigenetic heterogeneity (2014), Nat Methods, 11, 817-820), and transcriptomes (see e.g. Tang, F. et al. mRNA-Seq whole-transcriptome analysis of a single cell, (2009), Nat Methods, 6, 377-382) of single cells.

However, despite recent progress in detecting proteins by mass spectrometry with single-cell resolution (Budnik, B. et al, SCoPE-MS: mass spectrometry of single mammalian cells quantifies proteome heterogeneity during cell differentiation (2018), Genome Biol, 19, 161), it remains a major challenge to measure translation in individual cells.

Ribosome profiling can produce a snapshot of all the ribosomes active in a cell at a particular moment, i.e. generating a so-called translatome. Amongst others, ribosome profiling provides information on the location of translation start sites, the distribution of ribosomes on a messenger RNA, the speed of translating ribosomes, etc.

Ribosome profiling protocols have been described in e.g. Ingolia, N. T. et al, (Genome-wide analysis in vivo of translation with nucleotide resolution using ribosome profiling (2009), Science, 324, 218-223), Darnell, A. M. et al, (Translational Control through Differential Ribosome Pausing during Amino Acid Limitation in Mammalian Cells, (2018), Mol Cell, 71, 229-243) and Reid, D. W. et al (Simple and inexpensive ribosome profiling analysis of mRNA translation, (2015), Methods, 91, 69-74).

The existing methods for ribosome profiling however do not have sufficient sensitivity to measure the translation in individual cells. Such method would be very valuable to e.g. unravel disease mechanisms as well as to study the effects of drugs on protein translation in individual cells. Therefore, there is a strong need in the art for a more specific method to elucidate the translatome at a single-cell resolution.

### SUMMARY

The method of the invention can be summarized in the following embodiments:
Embodiment 1. A method for determining a translatome of a cell, comprising the steps of:
   i) lysing a single cell;
   ii) digesting the RNA with a ribonuclease, thereby generating a ribosome footprint containing RNA molecules that are protected against digestion;
   iii) Inactivating the ribonuclease and releasing the RNA molecules from the ribosomes;
   iv) end repairing the released RNA molecules;
   v) constructing an RNA library from the end-repaired RNA molecules;
   vi) size selecting part of the prepared RNA library for fragments having an insert size of about 20-40 nucleotides;
   vii) sequencing the size selected RNA library; and
   viii) determining the translatome of the cell, wherein preferably the cell is a single cell.
Embodiment 2. A method according to embodiment 1, wherein the ribonuclease in step ii) is a micrococcal nuclease (MNase).
Embodiment 3. A method according to embodiment 1 or 2, wherein in step iii) the ribonuclease is inactivated by a thermolabile proteinase K and/or the presence of a chelating agent.
Embodiment 4. A method according to embodiment 3, wherein the chelating agent is at least one of EDTA and EGTA.
Embodiment 5. A method according to any one of the preceding embodiments, wherein step iii) further comprises the presence of a chaotropic agent, wherein the chaotropic agent is preferably guanidium thiocyanite (GuSCN).
Embodiment 6. A method according to any of the preceding embodiments, wherein in step iv) a polynucleotide kinase (PNK) and a phosphate donor is used to end repair the released RNA molecules.
Embodiment 7. A method according to embodiment 6, wherein the phosphate donor is not ATP, preferably wherein the phosphate donor is selected from the group consisting of UTP, CTP, GTP, TTP, dATP and dTTP.
Embodiment 8. A method according to any one of the preceding embodiments, wherein the translatome of two or more cells are determined.
Embodiment 9. A method according to embodiment 8, wherein the method comprises a step of pooling the constructed RNA libraries after step v) and before step vi).
Embodiment 10. A method according to any one of the preceding embodiments, wherein the library preparation step v) comprises the sub-steps of:
   a) ligating a first adapter to the 3′-end and a second adapter to the 5′-end of the end-repaired RNA molecules, wherein preferably at least one of the first and second adapter comprises at least one of an UMI and a barcode;
   b) reverse transcribing the adapter-ligated RNA molecules to obtain cDNA; and
   c) amplifying the cDNA with a first and a second primer, wherein preferably at least one of first and second primer comprises a barcode.
Embodiment 11. A method according to embodiment 10, wherein the barcode in step a) and/or step c) is at least one of a cell barcode, a sample barcode and a plate barcode.
Embodiment 12. A method according to embodiment 10 or 11, wherein sub-step a) of ligating the first and/or second adapter is performed at a temperature below about 10° C., preferably at a temperature of about 4° C., preferably for a time period of at least 0.5, 1, 2, 4, 6, 8, 10, 12, 14 or 16 hours.
Embodiment 13. A method according to any one of embodiments 10-12, wherein sub-step a) of ligating the first and/or second adapter is performed in a buffer comprising polyethylene glycol (PEG), preferably PEG-8000, wherein the concentration PEG is preferably about 30%-40%, preferably about 30%, 31%, 32%, 33%, 34%, 35%, 36%, 37%, 38%, 39% or 40% or preferably about 15%-25%, preferably about 15%, 16%, 17%, 18%, 19%, 20%, 21%, 22%, 23%, 24% or 25%.
Embodiment 14. A method according to any one of embodiments 10-13 further comprising a complexity reduction step, wherein the complexity reduction step is preferably an amplification step d), wherein at least one of the primers comprises a selective nucleotide at the 3′-end for amplification of a subset of nucleotides.
Embodiment 15. A method according to any one of the preceding embodiments, wherein the cell is a mammalian cell, preferably a human cell, preferably a human tumor cell or an embryonic cell.
Embodiment 16. A method according to any one of the preceding embodiments, wherein the method does not comprise an RNA purification step.
Embodiment 17. A kit for use in the method of embodiments 1-16, wherein the kit comprises:
   i) a Ribonuclease, preferably a micrococcal nuclease;
   ii) a Polynucleotide kinase (PNK); and
   iii) at least one of UTP, CTP, GTP, TTP, dATP and dTTP.

### DETAILED DESCRIPTION

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al.. Molecular Cloning. A Laboratory Manual, 2nd Edition, Cold Spring Harbor Laboratory Press, Cold Spring Harbor, N. Y., 1989; Ausubel et al. Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. Thus, for example, reference to “a cell” includes a combination of two or more cells, and the like.

As used herein, the term “about” is used to describe and account for small variations. For example, the term can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“And/or”: the term “and/or” refers to a situation wherein one or more of the stated cases may occur, alone or in combination with at least one of the stated cases, up to with all of the stated cases.

As used herein, the term “adapter” is a single-stranded, double-stranded, partly double-stranded, Y-shaped or hairpin nucleic acid molecule that can be attached, preferably ligated, to the end of other nucleic acids, e.g., to a single strand of a RNA or DNA molecule, and preferably has a limited length, e.g., about 10 to about 200, or about 10 to about 100 bases, or about 10 to about 80, or about 10 to about 50, or about 10 to about 30 base pairs in length, and is preferably chemically synthesized. The double-stranded structure of the adapter may be formed by two distinct oligonucleotide molecules that are base paired with one another, or by a hairpin structure of a single oligonucleotide strand. As would be apparent, the attachable end of an adapter may be designed to be compatible with, and optionally able to ligate to, overhangs made by cleavage by a restriction enzyme and/or programmable nuclease, may be designed to be compatible with an overhang created after addition of a non-template elongation reaction (e.g. using the method as defined herein), or may have blunt ends. Optionally, the fully or partially double-stranded adapter comprises an overhang, wherein preferably the overhang is a 3′ overhang. Preferably, there is a phosphorothioate bond before the terminal nucleotide. Optionally, the strand opposite to the strand comprising the overhang, is 5′-phosphorylated. The adapter may comprise a modification such as a dideoxycytidine (ddC) modification or a terminal amino group, e.g. at the 3′-end, to prevent self-ligation.

“Amplification” used in reference to a nucleic acid or nucleic acid reactions, refers to in vitro methods of making copies of a particular nucleic acid, such as a target nucleic acid fragment or the sequence of interest comprised in the target nucleic acid fragment. Numerous methods of amplifying nucleic acids are known in the art, and amplification reactions include polymerase chain reactions, ligase chain reactions, strand displacement amplification reactions, rolling circle amplification reactions, transcription-mediated amplification methods such as NASBA (e.g., U.S. Pat. No. 5,409,818), loop mediated amplification methods (e.g., “LAMP” amplification using loop-forming sequences, e.g., as described in U.S. Pat. No. 6,410,278) and isothermal amplification reactions. The nucleic acid that is amplified can be DNA comprising, consisting of, or derived from, DNA or RNA or a mixture of DNA and RNA, including modified DNA and/or RNA. The products resulting from amplification of a nucleic acid molecule or molecules (i.e., “amplification products”), whether the starting nucleic acid is DNA, RNA or both, can be either DNA or RNA, or a mixture of both DNA and RNA nucleosides or nucleotides, or they can comprise modified DNA or RNA nucleosides or nucleotides.

A “copy” can be, but is not limited to, a sequence having full sequence complementarity or full sequence identity to a particular sequence. Alternatively, a copy does not necessarily have perfect sequence complementarity or identity to this particular sequence, e.g. a certain degree of sequence variation is allowed. For example, copies can include nucleotide analogs such as deoxyinosine or deoxyuridine, intentional sequence alterations (such as sequence alterations introduced through a primer comprising a sequence that can be hybridized, but is not complementary, to a particular sequence), and/or sequence errors that occur during amplification.

The term “complementarity” is herein defined as the sequence identity of a sequence to a fully complementary strand (e.g. the second, or reverse, strand). For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

“Comprising”: this term is construed as being inclusive and open ended, and not exclusive. Specifically, the term and variations thereof mean the specified features, steps or components are included. These terms are not to be interpreted to exclude the presence of other features, steps or components.

“The terms “double-stranded” and “duplex” as used herein, describes two complementary polynucleotides that are base-paired, i.e., hybridized together. Complementary nucleotide strands are also known in the art as reverse-complement.

The term “effective amount,” as used herein, refers to an amount of a biologically active agent or reaction enzyme that is sufficient to elicit a desired biological effect. For example, in some embodiments, an effective amount of a ribonuclease may refer to the amount of the nuclease that is sufficient to induce cleavage of an RNA molecule. As will be appreciated by the skilled artisan, the effective amount of an agent may vary depending on various factors such as the agent being used, the conditions wherein the agent is used, and the desired biological effect, e.g. degree of cleavage to be detected.

“Exemplary”: this term means “serving as an example, instance, or illustration,” and should not be construed as excluding other configurations disclosed herein.

“Expression”: this refers to the process wherein a DNA region, which is operably linked to appropriate regulatory regions, particularly a promoter, is transcribed into an RNA, which in turn may be translated into a protein or peptide.

The term “nucleotide” includes, but is not limited to, naturally-occurring nucleotides, including guanine, cytosine, adenine, thymine and uracil (G, C, A, T and U, respectively). The term “nucleotide” is further intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “nucleic acid”, “polynucleotide” and “nucleic acid molecule” are used interchangeably herein to describe a polymer of any length, e.g., greater than about 2 nucleotides, greater than about 10 nucleotides, greater than about 100 nucleotides, greater than about 500 nucleotides, greater than 1000 nucleotides, up to about 10,000 or more nucleotides, e.g., deoxyribonucleotides or ribonucleotides, and may be produced enzymatically or synthetically (e.g., PNA as described in U.S. Pat. No. 5,948,902 and the references cited therein). The nucleic acid may hybridize with naturally occurring nucleic acids in a sequence specific manner analogous to that of two naturally occurring nucleic acids, e.g., can participate in Watson-Crick base pairing interactions. In addition, nucleic acids and polynucleotides may be isolated (and optionally subsequently fragmented) from cells, tissues and/or bodily fluids. The nucleic acid can be e.g. an RNA molecule, DNA from a library and/or RNA from a library. The RNA molecule can be a coding or non-coding RNA molecule, and non-limiting examples of RNA molecules include, but not limited to, mRNA (fragment), pre-mRNA (fragment) and non-coding RNA. Preferably the RNA molecule is a (fragment of) an mRNA molecule.

The term “nucleic acid sample” as used herein denotes any sample containing a nucleic acid molecule, wherein a sample relates to a material or mixture of materials, typically, although not necessarily, in liquid form. The nucleic acid sample used as starting material in the method of the invention can be from any source, e.g., from one or more cells. transcribed genes. The nucleic acid samples can be obtained from the same individual, which can be a human or other species (e.g., plant, bacteria, fungi, algae, archaea, etc.), or from different individuals of the same species, or different individuals of different species. For example, the nucleic acid samples may be from a cell, tissue, biopsy, bodily fluid, genome DNA library, cDNA library and/or an RNA library.

The term “oligonucleotide” as used herein denotes a single-stranded multimer of nucleotides, preferably of about 2 to 200 nucleotides, or up to 500 nucleotides in length. Oligonucleotides may be synthetic or may be made enzymatically, and, in some embodiments, are about 10 to 50 nucleotides in length. Oligonucleotides may contain ribonucleotide monomers (i.e., may be oligoribonucleotides) or deoxyribonucleotide monomers. An oligonucleotide may be about 10 to 20, to 30, 30 to 40, 40 to 50, 50 to 60, 60 to 70, 70 to 80, 80 to 100, 100 to 150, 150 to 200, or about 200 to 250 nucleotides in length, for example.

“Reducing complexity” or “complexity reduction” is to be understood herein as the reduction of a complex nucleic acid sample, such as samples derived from genomic DNA, cfDNA derived from liquid biopsies, isolated RNA samples and the like. Reduction of complexity results in the enrichment of one or more specific target sequences and/or target nucleic acid fragments comprised within the complex starting material and/or the generation of a subset of the sample, wherein the subset comprises or consists of one or more specific target sequences or fragments comprised within the complex starting material, while non-target sequences or fragments are reduced in amount by at least 20%, 30%, 40%, 50%, 60%, 70%, 80%, 90%, 91%, 92%, 93%, 94%, 95%, 96%, 97%, 98% or 99% as compared to the amount of non-target sequences or fragments in the starting material, i.e. before complexity reduction. Reduction of complexity is in general performed prior to further analysis or method steps, such as amplification, barcoding, sequencing, determining epigenetic variation etc. Preferably, complexity reduction is reproducible complexity reduction, which means that when the same sample is reduced in complexity using the same method, the same, or at least comparable, subset is obtained, as opposed to random complexity reduction. Examples of complexity reduction methods include for example Arbitrarily Primed PCR amplification, capture-probe hybridization, the methods described by Dong (see e.g., WO 03/012118, WO 00/24939) and indexed linking (Unrau P. and Deugau K. V. (1994) Gene 145:163-169), the methods described in WO2006/137733; WO2007/037678; WO2007/073165; WO2007/073171, US 2005/260628, WO 03/010328, US 2004/10153, genome portioning (see e.g. WO 2004/022758), Serial Analysis of Gene Expression (SAGE; see e.g. Velculescu et al., 1995, see above, and Matsumura et al., 1999, The Plant Journal, vol. 20 (6): 719-726) and modifications of SAGE (see e.g. Powell, 1998, Nucleic Acids Research, vol. 26 (14): 3445-3446; and Kenzelmann and MOhlemann, 1999, Nucleic Acids Research, vol. 27 (3): 917-918), MicroSAGE (see e.g. Datson et al., 1999, Nucleic Acids Research, vol. 27 (5): 1300-1307), Massively Parallel Signature Seguencing (MPSS; see e.g. Brenner et al., 2000, Nature Biotechnology, vol. 18:630-634 and Brenner et al., 2000, PNAS, vol. 97 (4):1665-1670), self-subtracted cDNA libraries (Laveder et al., 2002, Nucleic Acids Research, vol. 30(9):e38), Real-Time Multiplex Ligation-dependent Probe Amplification (RT-MLPA; see e.g. Eldering et al., 2003, vol. 31 (23): e153), High Coverage Expression Profiling (HiCEP; see e.g. Fukumura et al., 2003, Nucleic Acids Research, vol. 31(16) :e94), a universal micro-array system as disclosed in Roth et al.(Roth et al., 2004, Nature Biotechnology, vol. 22 (4): 418-426), a transcriptome subtraction method (see e.g. Li et al., Nucleic Acids Research, vol. 33 (16): e136), and fragment display (see e.g. Metsis et al., 2004, Nucleic Acids Research, vol. 32 (16): e127).

“Sequence” or “Nucleotide sequence”: This refers to the order of nucleotides of, or within a nucleic acid. In other words, any order of nucleotides in a nucleic acid may be referred to as a sequence or nucleic acid sequence. For example, the target sequence is an order of nucleotides comprised in an RNA or DNA molecule.

The term “sequencing” as used herein, refers to a method by which the identity of at least 10 consecutive nucleotides (e.g., the identity of at least 20, at least 50, at least 100 or at least 200 or more consecutive nucleotides) of a polynucleotide are obtained. The terms “next-generation sequencing”, “deep-sequencing” or “high-throughput sequencing” may be used interchangeably herein and refers to the so-called parallelized sequencing-by-synthesis or sequencing-by-ligation platforms, e.g., such as currently employed by Illumina, Life Technologies, PacBio and Roche etc. Next-generation sequencing methods may also include nanopore sequencing methods, such as those commercialized by Oxford Nanopore Technologies, or electronic-detection based methods such as Ion Torrent technology commercialized by Life Technologies.

A “barcode” is defined herein as a sequence of varying length that is used to distinguish a nucleic acid from a second or further nucleic acid. The length of a barcode is preferably between 2-20, 5-15, or between about 7-10 nucleotides. The barcode preferably does not comprise two or more identical adjacent nucleotides. The barcode may at least one of a sample barcode, a cell barcode, a plate barcode or a UMI.

A “unique molecular identifier” or “UMI” is a substantially unique tag (e.g. barcode), preferably fully unique, that is specific for a nucleic acid molecule, e.g. unique for each single polynucleotide. The term “UMI” is used herein to refer to both the sequence information of a polynucleotide and the physical polynucleotide per se. A UMI can range in length from about 2 to 100 nucleotide bases or more, and preferably has a length between about 4-16 nucleotide bases. The UMI can be a consecutive sequence or may be split into several subunits. Each of these subunits may be present in separate oligonucleotides and/or adapters. These subunits are preferably used together to generate a substantially unique tag, preferably a fully unique tag, for a single polynucleotide. For instance, if a polynucleotide is a fragment flanked by two oligonucleotides, each of these two oligonucleotides may comprise a subunit of the UMI. In case the polynucleotide is a ligation product of two oligonucleotides, each of these two oligonucleotides may comprise a subunit of the UMI. In order to obtain a consensus sequence, the sequence reads obtained in the method of the invention may be grouped based on the information of each of the two UMI subunits. Preferably a UMI does not contain two or more consecutive identical bases. Furthermore, there is preferably a difference between UMIs of at least two, preferably at least three bases. A UMI may have random, pseudo-random or partially random, or a non-random nucleotide sequence. As a UMI can be used to uniquely identify the originating molecule from which the read is derived, reads of amplified polynucleotides can be collapsed into a single consensus sequence from each originating polynucleotide. A UMI may be fully or substantially unique. Fully unique is to be understood herein as that every polynucleotide provided in the method of the invention comprises a unique tag that differs from all the other tags comprised in further polynucleotides in the method of the invention. Substantially unique is to be understood herein in that each polynucleotide provided in the method, product, composition or kit of the invention comprises a random UMI, but a low percentage of these polynucleotides may comprise the same UMI. Preferably, substantially unique molecular identifiers are used in case the chances of tagging the exact same molecule comprising the sequence of interest with the same UMI is negligible. Preferably, a UMI is fully unique in relation to a specific sequence of interest. A UMI preferably has a sufficient length to ensure this uniqueness. In some implementations, a less unique molecular identifier (i.e. a substantially unique identifier, as indicated above) can be used in conjunction with other identification techniques to ensure that each DNA molecule is uniquely identified during the sequencing process. For instance, the UMI of the invention may be less unique such that different sequences of interest may be coupled to the same or similar UMI. In the latter case, the combination of the sequence information of the UMI together with the sequence information of the sequence of interest allows for the identification of the originating polynucleotide. A UMI is preferably used to determine that all reads from a single cluster are identified as deriving from a single molecule.

A “translatome” is defined herein as the total of mRNA fragments that are translated at a certain point in time in a single cell.

The inventors discovered a method that majorly increases the sensitivity of existing ribosome profiling protocols, thereby allowing ribosome profiling in single cells. This method of the invention achieves single codon resolution in individual cells. As shown in the examples below, the method of the invention is used to demonstrate that limitation for a particular amino acid causes ribosome pausing at a subset of the codons representing this amino acid. This pausing was only observed in a sub-population of cells correlating to its cell-cycle state. The method was further used to detect pronounced GAA pausing during mitosis in non-limiting conditions. Furthermore, this method was used to measure ribosome profiles in primary mouse enteroendocrine cells. This new technology thus provides the first steps towards determining the contribution of the translational process to the astonishing diversity between seemingly identical cells.

The method of the invention can be used to discover changes in the translation of particular mRNAs, such as changes in the translation rate or the preferred translation of transcript isoforms in single cells. This provides for a novel valuable approach to unravel disease mechanisms. Similarly, determining the translatome of the single cells aids in determining the effects of drug compounds on these single cells.

The method of the invention (scRibo-seq) combines nuclease footprinting with small RNA library construction and a size enrichment to measure translation in single cells (FIG. 1a). Briefly, single live cells are first sorted into a lysis buffer to stabilize and halt ribosomes on transcripts. Exposed RNA is then digested by micrococcal nuclease (MNase) and the resulting ribosome-protected footprints (RPFs) are then released. These footprints are converted into sequencing libraries by ligating adaptors that contain a unique molecular identifier (UMI) and priming sites for subsequent cDNA synthesis and indexing PCR. Finally, the reaction products from each cell are pooled and size selected to enrich for inserts that correspond to the typical ribosome footprint length. These steps are now all combined into one efficient workflow, obviating the need of intermediate purification and clean-up steps, which steps would lead the inevitable loss of nucleic acid material. As a result, it is now feasible to perform ribosome profiling on single cells.

The method as detailed herein is a method for determining a translatome of a single cell. The method can equally be considered:
a method for single cell ribosome profiling; and/or
a method for generating a sequencing library from the translatome of a single cell; The method as detailed herein can further be a method for determining the effects of a compound, such as a therapeutic drug, on the translatome of a single cell. In such method, the method as detailed herein may be preceded by a step of exposing the cell to a compound under suitable conditions, prior to lysing the cell.

In an aspect, the method of the invention is a method for determining a translatome of a cell, comprising the steps of:
i) lysing a single cell;
ii) digesting the RNA with a ribonuclease, thereby generating a ribosome footprint containing RNA molecules that are protected against digestion;
iii) Inactivating the ribonuclease and releasing the RNA molecules from the ribosomes;
iv) end repairing the released RNA molecules;
v) constructing an RNA library from the end-repaired RNA molecules;
vi) optionally, size selecting part of the prepared RNA library for fragments having an insert size of about 20-40 nucleotides;
vii) sequencing the, optionally size selected, RNA library; and
viii) determining the translatome of the cell.

Preferably, the cell is a single cell. The single cell may be isolated e.g. using conventional FACS sorting.

Preferably, the RNA library is so-called “small RNA library”.

Preferably, the ribonuclease in step ii) is selected from the group consisting of MNase, RNase I RNase A and RNase T1, or any combination thereof. Preferably, the ribonuclease in step ii) is a micrococcal nuclease (MNase).

Preferably, in step iii) the ribonuclease is inactivated by a thermolabile proteinase, preferably a thermolabile proteinase K, and/or the presence of a chelating agent.

Preferably, the chelating agent is at least one of EDTA and EGTA.

Preferably, step iii) further comprises the presence of a chaotropic agent, wherein the chaotropic agent is preferably guanidium thiocyanite (GuSCN).

Preferably, step iv) a polynucleotide kinase (PNK) and a phosphate donor is used to end repair the released RNA molecules. Preferably, the make them compatible with the library construction steps as detailed herein.

The phosphate donor is preferably not ATP. Preferably, the phosphate donor is selected from the group consisting of UTP, CTP, GTP, TTP, dATP and dTTP, preferably UTP (uridine triphosphate).

In a preferred method, the translatome of two or more cells are determined.

In case the method is performed on multiple samples, the method preferably comprises a step of pooling the constructed RNA libraries after step v) and before step vi).

Preferably, the library preparation step v) comprises the sub-steps of:
a) ligating a first adapter to the 3′-end and a second adapter to the 5′-end of the end-repaired RNA molecules, wherein preferably at least one of the first and second adapter comprises at least one of an UMI and a barcode;
b) reverse transcribing the adapter-ligated RNA molecules to obtain cDNA; and
c) amplifying the cDNA with a first and a second primer, wherein preferably at least one of first and second primer comprises a barcode.

Preferably, the barcode in step a) and/or step c) is at least one of a cell barcode, a sample barcode and a plate barcode.

In a preferred method, sub-step a) of ligating the first and/or second adapter is performed at a temperature below about 10° C., preferably at a temperature of about 4° C., preferably for a time period of at least about 0.5, 1, 2, 4, 6, 8, 10, 12, 14 or 16 hours.

Preferably, in sub-step a) the ligation the first and/or second adapter is performed in a buffer comprising polyethylene glycol (PEG), preferably PEG-8000, wherein the concentration PEG is preferably about 30%-40%, preferably about 30%, 31%, 32%, 33%, 34%, 35%, 36%, 37%, 38%, 39% or 40% or preferably about 15%-25%, preferably about 15%, 16%, 17%, 18%, 19%, 20%, 21%, 22%, 23%, 24% or 25%.

Preferably, the library preparation further comprises a complexity reduction step, wherein the complexity reduction step is preferably an amplification step d), wherein at least one of the primers comprises a selective nucleotide at the 3′-end for amplification of a subset of nucleotides.

The cell for use in the method of the invention is preferably a mammalian cell, preferably a human cell, preferably a human tumor cell or an embryonic cell.

The method of the invention preferably does not comprise an RNA purification step. Preferably, the method does not comprise the use of e.g. Trizol for RNA purification.

Alternatively or in addition, the method of the invention preferably does not comprise a step of monosome purification. Preferably, the method does not comprise a sucrose gradient purification step.

In a further aspect, the invention pertains to a kit for use in the method of the invention. Preferably, the kit comprises at least three components selected from the group consisting of:
i) a Ribonuclease, preferably a micrococcal nuclease;
ii) a Polynucleotide kinase (PNK);
iii) at least one of UTP, CTP, GTP, TTP, dATP and dTTP;
iv) A thermolabile protease;
v) a chelating agent, preferably at least one of EDTA and EGTA;
vi) a chaotrope, preferably guanidium thiocyanite (GuSCN);
vii) T4 RNA ligase 2, preferably truncated, preferably mutated and truncated;
viii) T4 RNA ligase 1;
ix) 3′ adapter; DNA, preferably at least one of 5′ adenylated and 3′ blocked (preferably blocked with at least one of Dideoxycytidine (ddC) and amino/NH2)
x) 5′ adapter;
xi) Reverse transcriptase, preferably in combination with an oligonucleotide to prime; and
xii) Thermostable DNA polymerase+primers

Optionally, the kit comprises at least the following components:
i) a Ribonuclease, preferably a micrococcal nuclease;
ii) a Polynucleotide kinase (PNK); and
iii) at least one of UTP, CTP, GTP, TTP, dATP and dTTP;

The reagents may be present in lyophilized form, or in an appropriate buffer. The kit may also contain any other component necessary for carrying out the present invention, such as buffers, pipettes, microtiter plates and written instructions. Such other components for the kits of the invention are known to the skilled person.
The present invention has been described above with reference to a number of exemplary embodiments. Modifications and alternative implementations of some parts or elements are possible, and are included in the scope of protection as defined in the appended claims.

### FIGURE LEGENDS

FIG. 1 scRibo-seq measures translation in singe cells. a. scRibo-seq method. b. Heatmap of the fold change of the number of 5′ cuts in regions around the start codon (left), in the coding sequence (middle), and around the stop codon (right). c. Length-corrected distribution of 5′ cuts across the 5′ UTR, CDS, and 3′ UTR. d.. Frame and read-length distributions of the 5′ end of RPFs and random-forest predicted P-sites averaged across cell types and e. in single cells. f. Number of footprints per cell along a metagene region within coding sequences before (1F.1: reads whose 5′ ends align at the given region) and after (1 F.2, number of predicted P-sites at each location) the random-forest correction.

FIG. 2 Ribosome pausing under amino acid limitation. a. Pseudobulk analysis of codon occupancy in ribosome E, P, and A sites. b. Heatmap of the fold change in codon occupancy in sites around the ribosome active sites. c. UMAP of the single-cell RPF libraries showing limitation condition and clusters. d. UMAPs showing the mean log 2 fold change in occupancy for arginine and leucine codons. e. Bar chart of the average of the P-site occupancy along a section of H3C2 for cells sorted and grouped based on their global arginine pausing. f. Heatmap showing RPF counts per coding sequence of the top marker genes for each cell cluster. g. Heatmap of the single-cell P-site occupancy along H3C2.

FIG. 3 Comparison to bulk methods for ribosome profiling. a. Region-length normalized distributions of RPF mapping frequencies in the 5′ UTR, CDS, and 3′ UTR regions of protein-coding transcripts. In the boxplots the middle line indicates the median, the box limits the first and third quartiles, and the whiskers the range. Lengths were determined assuming all RPFs originated from the same transcript. b. Fraction of reads per library across a scaled metagene for six bulk ribosome profiling libraries generated on RPE1 cells. Data from Tanenbaum, M. E., Stern-Ginossar, N., Weissman, J. S. & Vale, R. D. Regulation of mRNA translation during mitosis. Elife 4, (2015).

FIG. 4 Random forest model corrects MNase sequence bias. a. Sequence logos around the 5′ and 3′ cut location. b. Truth table for the validation data. c. Permutation importance of the model features.

FIG. 5 Ribosome pausing in single cells. a. Heatmap of log 2 fold change of respective amino acid occupancy in the RPF reads. b. Distribution of cells exhibiting ribosome pausing in clusters. The threshold used to distinguish pausing cells was calculated as the mean plus two standard deviations of the signal of the cells from the rich condition.

FIG. 6 Ribosome pausing during the cell cycle. a-e. UMAPs (n=1777 cells) illustrating the a. cell fractions, b. cell clusters, c. pseudotime trajectory, and d. fluorescence of the mKO2-CDT1 and e. mAG-GMNN FUCCI markers. f-h. Scatterplot of the FUCCI markers (n=1777 cells) denoting the f. cell fractions, g. cell clusters, and h. pseudotime trajectory. i. Heatmap showing the site-specific pausing in single cells ordered based on cell-cycle progression. j. UMAP showing the GAA pausing and k. AUA pausing. I. Heatmap showing the positions of RPF A-sites along the MYL6 coding sequence. m. Scatterplots showing the fold change in gene-wise A-site frequency of occupancy between each cell cluster and the background.

FIG. 7 Heatmap showing translation dynamics of 1531 genes during the cell cycle, highlighting cell-cycle markers.

FIG. 8 Codon pausing during the cell cycle. a. Codon frequency of occurrence in each ribosome site along pseudotime. The upper and lower bounds of codon usage are shown on the right. b. Scatterplots showing the fold change in gene-wise A-site frequency of occupancy between each cell cluster and the background for the listed codons.

FIG. 9 Heatmap showing codon pausing during the cell cycle.

FIG. 10 Single-cell ribosome profiling in primary mouse intestinal enteroendocrine (EEC) cells. a. UMAP (n=350 cells) generated using the RPF counts per CDS. Corresponding cell types and associated marker genes for each cluster are indicated. b-c. UMAPs illustrating the fluorescence of the b. mNeonGreen and c. dTomato markers from the bi-fluorescent Neurog3 Chrono reporter (Gehart, H. et al. Identification of Enteroendocrine Regulators by Real-Time Single-Cell Differentiation Mapping. Cell 176, 1158-1173 e1116, (2019)). d. UMAP depicting the intestinal region origin of each cell. As expected, there is no enrichment of the cell types within each region. e. Scatterplots of the Neurog3 Chrono fluorescence denoting the position of each cell cluster within the FACS space. As expected, progenitor cells show an increased mNeonGreen fluorescence, that changes through a double-positive population to dTomato-positive as EEC cells develop. f. Heatmap showing ribosome-site-specific pausing over CAG and GAA codons. To remove any effects of the uneven distribution of RPFs along highly-translated hormone genes, any gene that was more than an average of 2.5% of the RPFs per cell was removed from this analysis. g-h. UMAPs showing the g. CAG and h. GAA pausing. i. Heatmap showing the distribution of RPF A-sites along the Chgb coding sequence. Cells are grouped based on their CAG and GAA pausing status. The position of CAG (orange) and GAA (purple) codons within the coding sequence are denoted as ticks at the top, with shared prominent pausing sites for each codon indicated with inverted triangles. j-k. Scatterplots showing the fold change in gene-wise A-site frequency of occurrence between the pausing and non-pausing (normal) cells within each cluster.

FIG. 11 Marker genes and codon pausing for enteroendocrine (EEC) cells. a. Heatmap of 1517 genes significantly differentially expressed between the cell clusters. Common EEC marker genes are indicated. b. UMAPs (n=350 cells) showing the expression of common EEC marker and hormone genes. c. Heatmap showing ribosome-site-specific pausing for all codons in the enteroendocrine cells. Cells are clustered based on the profiles across the codons. To remove any effects of the uneven distribution of RPFs along highly-translated hormone genes, any gene that was more than an average of 2.5% of the RPFs per cell was removed from this analysis (removed genes: Chga, Chgb, Clca1, Fcgbp, Gcg, Ghrl, Gip, Nts, Reg4, Sst).

FIG. 12 Comparison of MNase and RNase I in generating ribosome footprints for scRibo-seq. a. Library performance metrics comparing the fraction of unique protein-coding reads, CDS-aligned reads, and number of detected genes between titrations of MNase and RNase I. b. Scatterplot comparing the normalized read counts per gene between MNase and RNase I libraries. c. Fraction of reads aligning to transfer RNA (tRNA) and ribosomal RNA (rRNA) between titrations of MNase and RNase I. d. Percent of RPFs aligning in each frame. Dashed grey line indicates the percent of in-frame alignments (62.5%) for the experimental conditions used in scRibo-seq. e. Heatmap of the number of ribosome footprints that align along metagene regions around the start and stop codons. The relative mapping coordinate of the 5′ end of each read is reported.

FIG. 13 Comparison of scRibo-seq to conventional ribosomal profiling. a-b. Heatmaps of the percentage of protein-coding reads per library aligning along metagene regions around the start codon (left), in the coding sequence (middle), and around the stop codon (right). The mapping coordinate of the a. 5′ end, or b. the random-forest predicted P-site of each read is reported. Libraries are from this work (scRibo-seq), and representative bulk ribosomal profiling methods: Darnell, using MNase on HEK293T (Darnell, A. M et al, Mol Cell 71, 229-243 e211, 2018); Ingolia, using RNase Ion HEK293T (Ingolia, N. T., et al, Nat Protoc 7, 1534-1550, 2012); Martinez, using RNase I on HEK293T (Martinez, T. F. et al. Nat Chem Biol 16, 458-468, 2020); and Tanenbaum, using RNase I on RPE-1 (Tanenbaum, M. E. et al, Elife 4, eLife.07957, 2015). c. Frame and read-length distributions of the 5′ end of RPFs and random-forest predicted P-sites averaged across library sets. d. Distributions of the percentage of trimmed reads aligning to rRNA and tRNA. e. Region-length normalized distributions of RPF mapping frequencies in the 5′ UTR, CDS, and 3′ UTR regions of protein-coding transcripts. f. Distributions of the percentage of trimmed reads that uniquely align to protein coding, lncRNA, snoRNAs, or other biotypes. In the boxplots in d-f the middle line indicates the median, the box limits the first and third quartiles, and the whiskers the range. Each point is from a single-cell or bulk library. g. Comparisons of the RPF counts per coding sequence in HEK293T cells between the different studies. Spearman correlation coefficients for each comparison are indicated.

### EXAMPLES

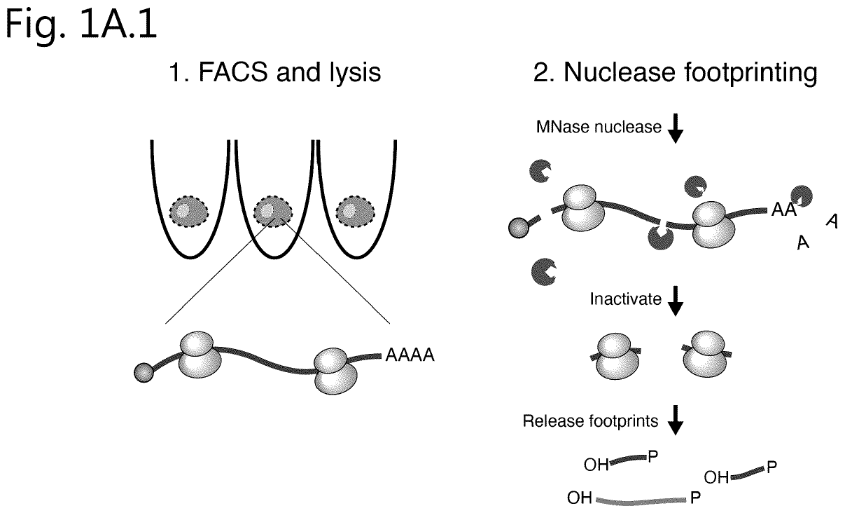

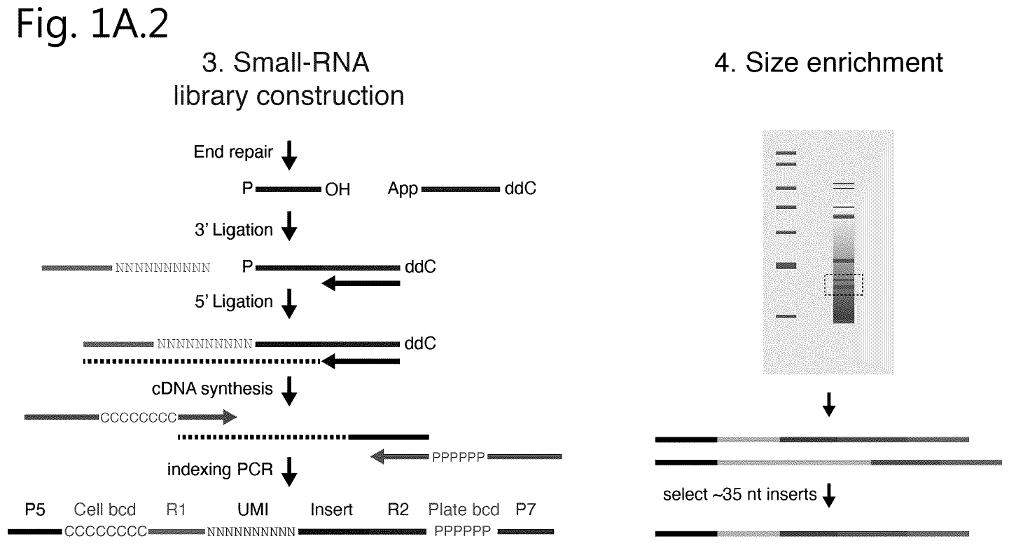

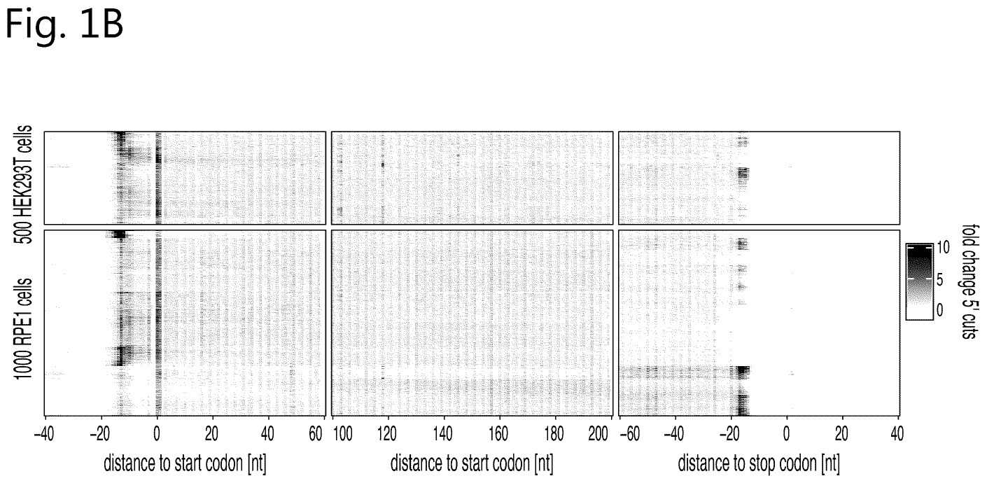

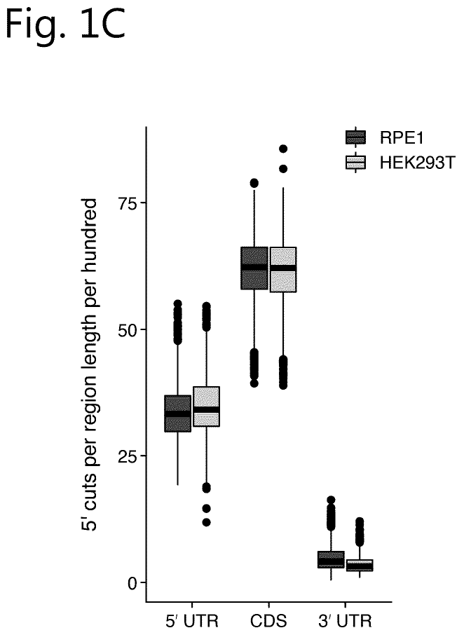

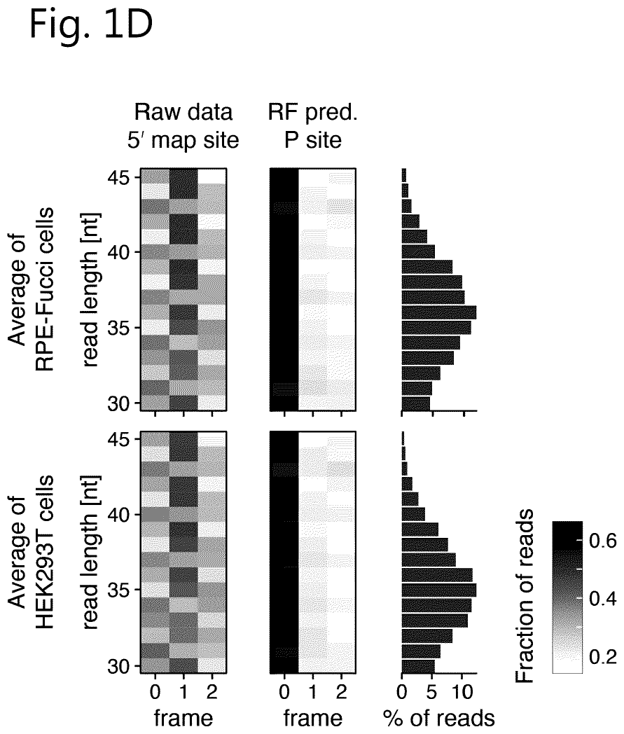

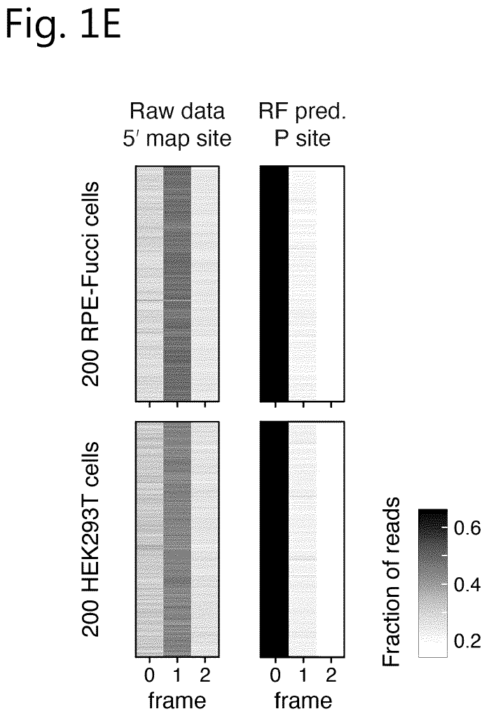

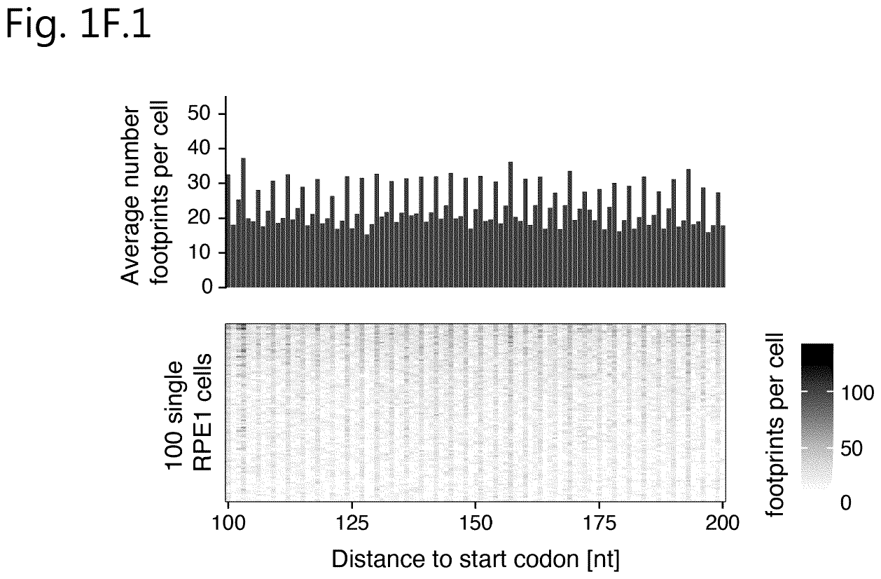

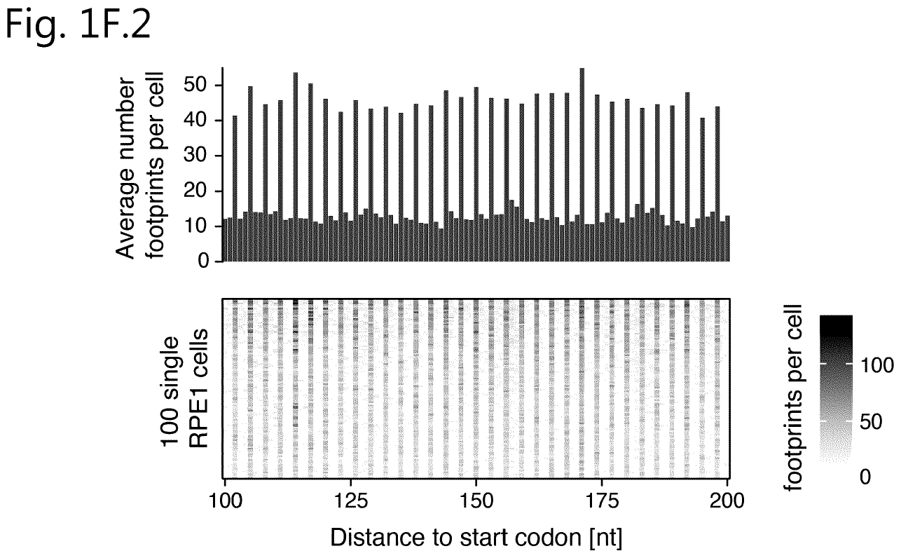

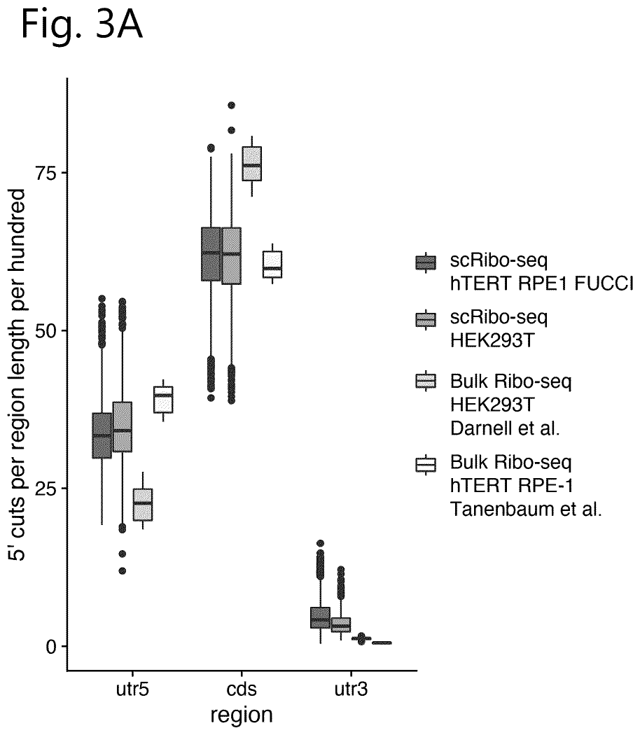

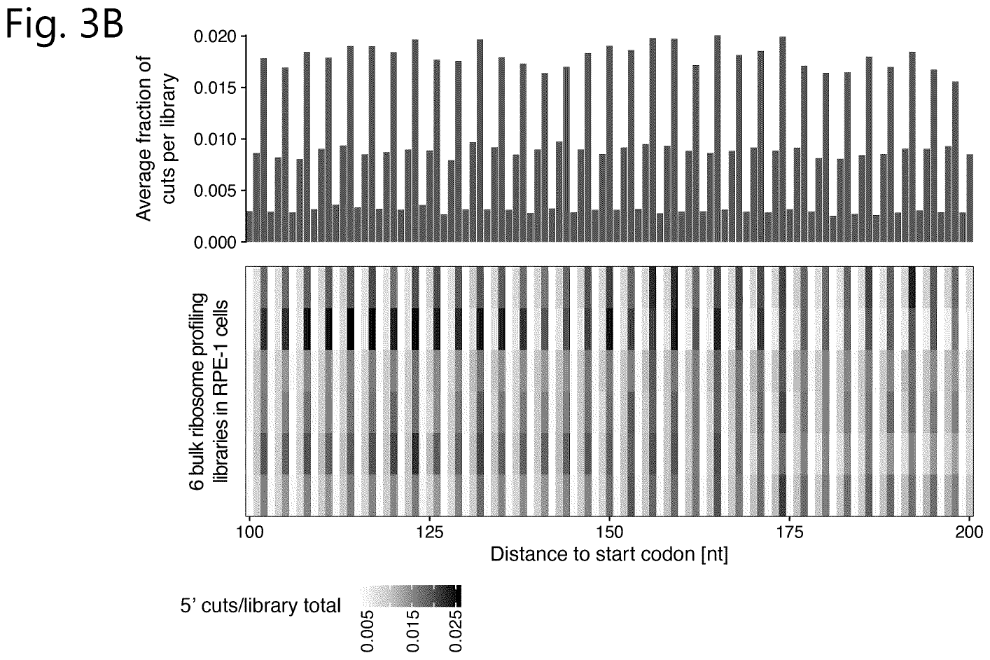

To validate this method, we generated scRibo-seq libraries from HEK293T and hTERT RPE-1 cells. The resulting single-cell libraries exhibit several features that are characteristic of ribosomal profiling experiments. First, the fragments predominantly map to coding sequences (FIG. 1b-c), with their 5′ ends sharply increasing ˜15 nucleotides upstream of the start codon and decreasing ˜18 nucleotides upstream of the stop codon (FIG. 1b, left and right panels). Additionally, the distribution of reads across the untranslated regions (UTR) and coding sequences (CDS) is similar to that from conventional ribosome profiling methods that explicitly purify monosomes (FIG. 3a, 13d-f). Second, there is an increase in local density over both the start and stop codons (FIG. 1b), originating from ribosomes that are in the initiation and termination phases of translation. Finally, the 5′ end of the fragments show a clear but modest 3-nucleotide periodicity along the coding sequence (FIG. 1b), with (41.5±1.9) % of the 5′ ends of the footprints occurring in frame 1 (FIG. 1e left).

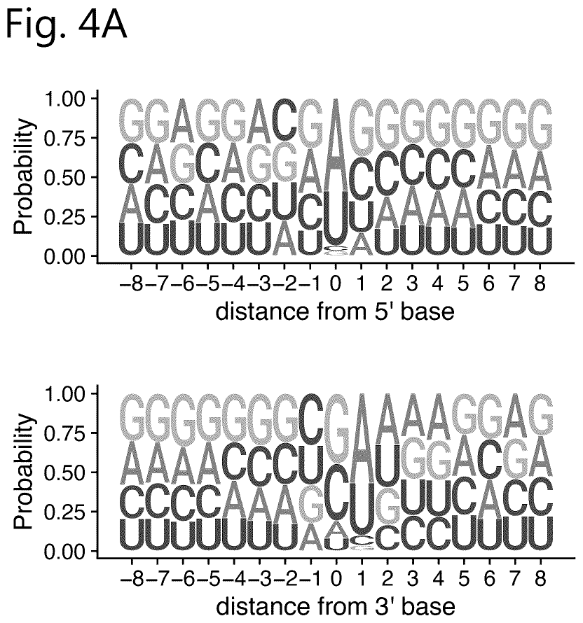

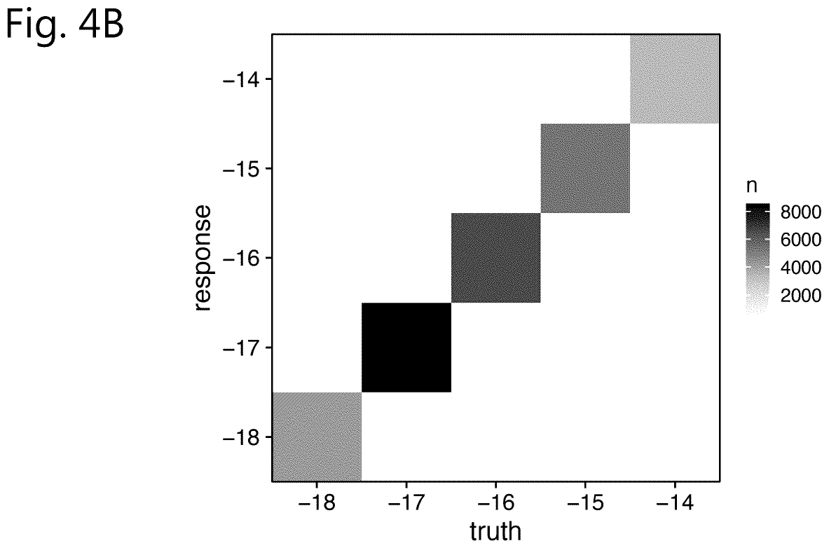

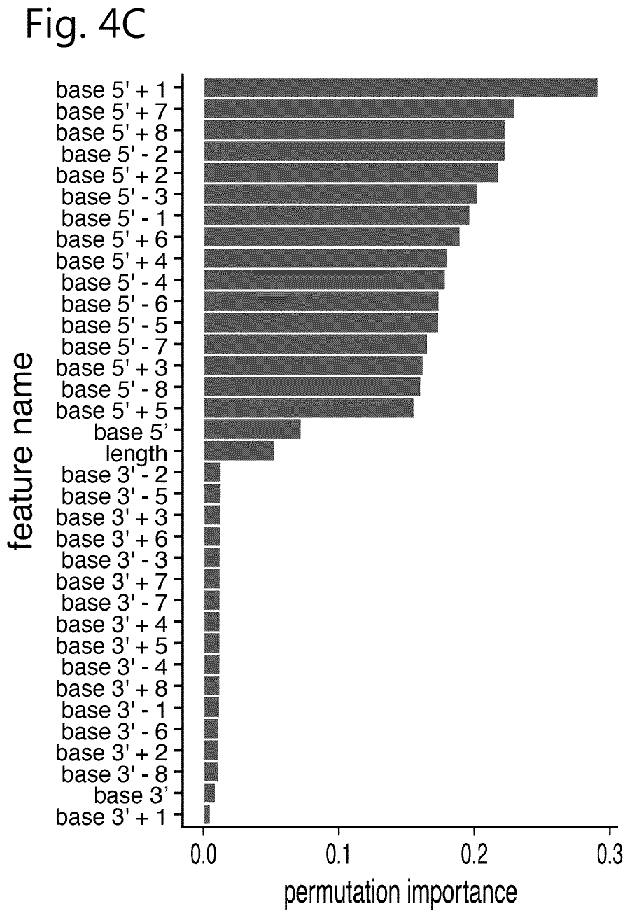

scRibo-seq libraries also display traits introduced by the MNase digestion. Consistent with previous reports (Darnell, A. M. et al, Translational Control through Differential Ribosome Pausing during Amino Acid Limitation in Mammalian Cells. (2018) Mol Cell, 71, 229-243; and Gerashchenko, M. V. & Gladyshev, V. N. Ribonuclease selection for ribosome profiling (2017), Nucleic Acids Res, 45), we observe a broad distribution of footprint lengths (FIG. 1d right), a complex association between fragment length and the predominant frame of the 5′ end (FIG. 1d left), and a strong preference for an MNase cut to occur to the 5′ of an adenine or uracil (FIG. 4a). We predicted that this strong sequence bias would result in incomplete digestion of the ribosome footprints, resulting in a sequence-dependent relationship between the 5′ and 3′ ends of the fragment and the active sites of the ribosome.

We trained a random forest (RF) classifier to correct for the MNase sequence bias. Similar to previous approaches (Fang, H. et al. Scikit-ribo Enables Accurate Estimation and Robust Modeling of Translation Dynamics at Codon Resolution (2018), Cell Syst, 6, 180-191), our model predicts the offset between the 5′ end of the footprint and the ribosome A-site given the length of the fragment and the sequence context around the 5′ and 3′ cut sites. The classifier was trained using only reads that spanned a stop codon, achieving a high prediction accuracy (mean accuracy (96.5±0.1) %, 5-fold CV; FIG. 4b). The accuracy was further confirmed by examining footprints within the CDS, where (63.6±1.0) % of predicted A-sites were found to be in frame (FIG. 1d,e,f), which was reproducible between cells (FIG. 1e right), and is again similar to that seen by conventional ribosome profiling methods on RPE1 cells ((60.5±6.7) %; FIG. 3b, 13a). As expected, the sequence composition around the 5′ end had the highest permutation importance amongst the classification features, followed by the fragment length, and only a minor contribution from the 3′ sequence context (FIG. 4c), suggesting that our model is indeed capturing the MNase sequence bias.

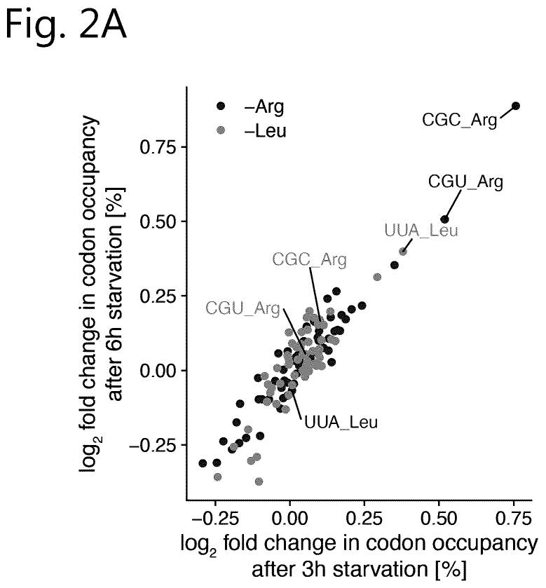

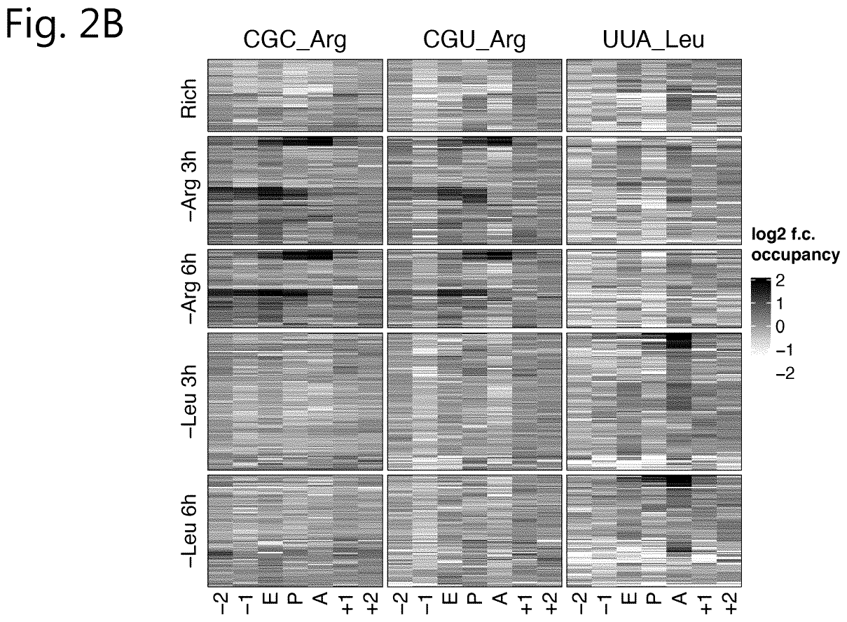

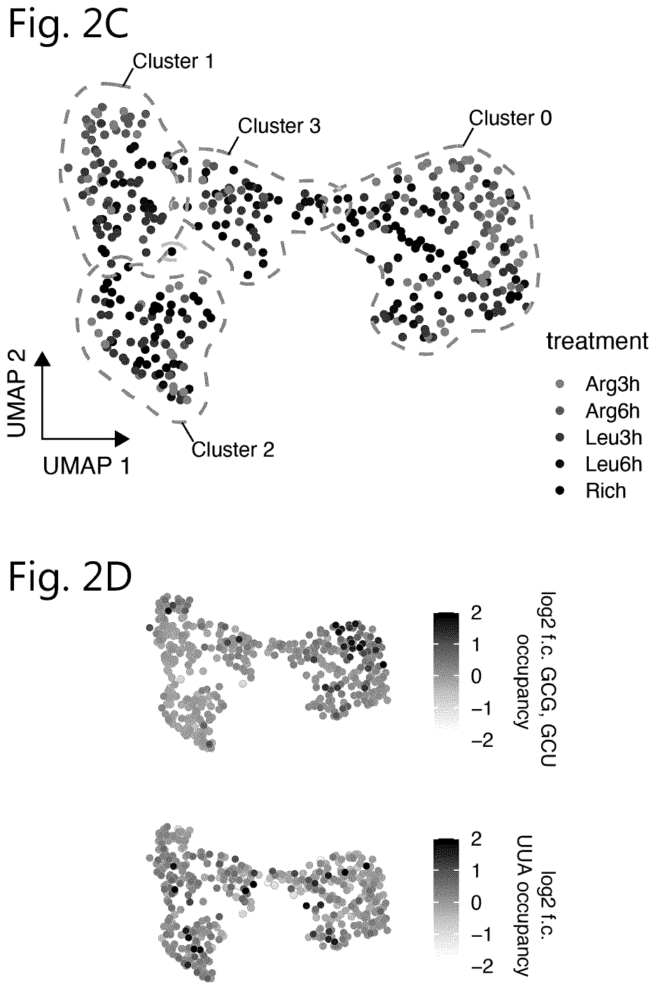

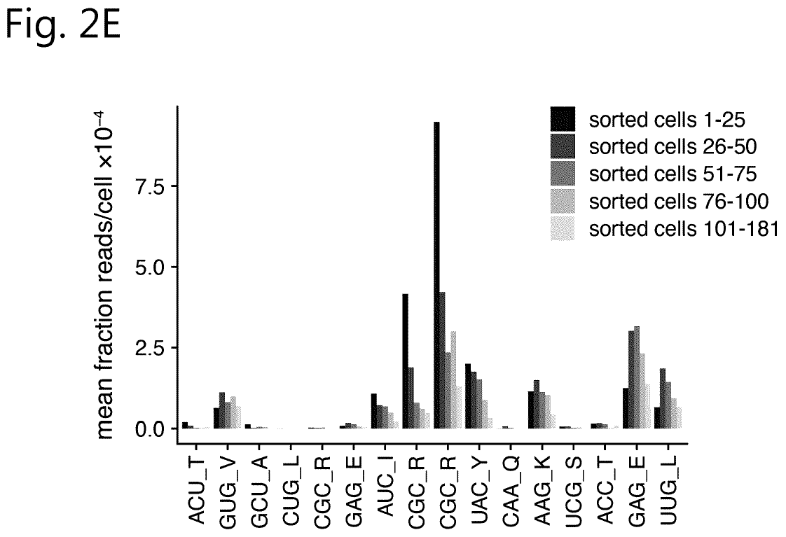

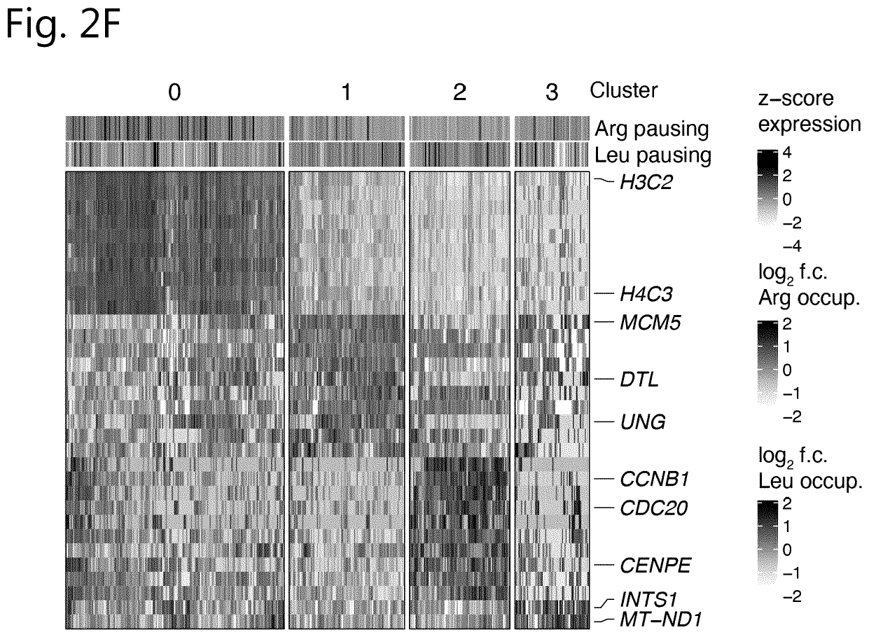

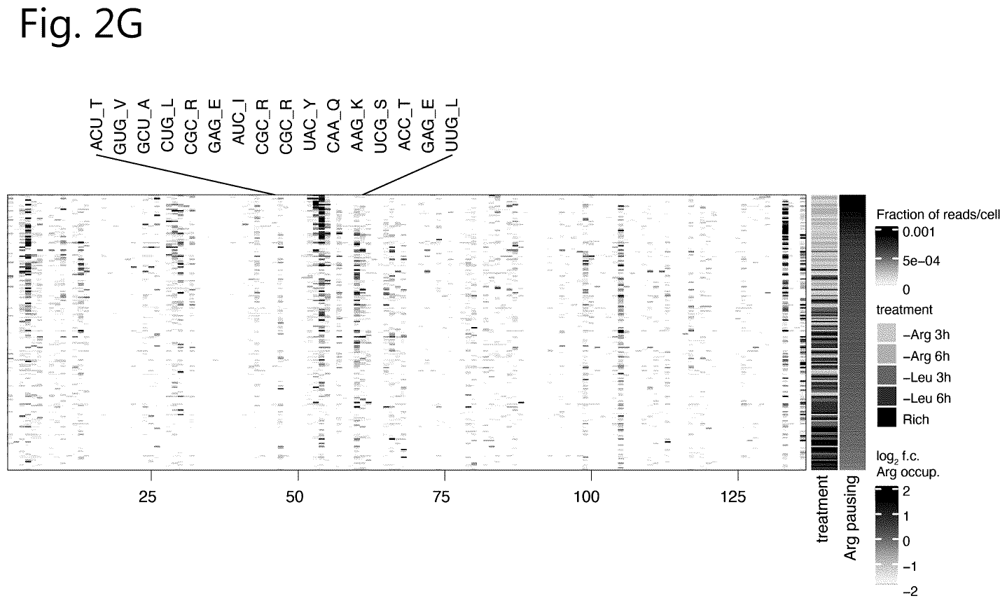

Ribosomes have been previously seen to dwell over a subset of codons encoding essential amino acids that have been removed from culture media (Darnel AM et al, supra; Subramaniam, A. R., Pan, T. & Cluzel, P. Environmental perturbations lift the degeneracy of the genetic code to regulate protein levels in bacteria. Proc Natl Acad Sci USA (2013), 110, 2419-2424). Ribosome profiling exposes this pausing as an increase in footprint density over the affected codons. To further validate that scRibo-seq measures translation dynamics, we cultured cells under amino acid starvation conditions. Arginine and leucine were each removed from HEK293T culture media for 3 and 6 hours before making scRibo-seq libraries. By comparing the change in codon occupancy in the predicted E, P, and A-sites between pseudobulks of the depletion and rich conditions, we observe treatment-specific pausing (FIG. 2a). For example, arginine depletion results in footprints more frequently residing over CGC and CGU codons compared to rich media (FIG. 2a, dark grey), and this increase is not seen upon leucine removal (FIG. 2a, light grey). Similarly, an increase in UUA occupancy is only seen in leucine starvation conditions.

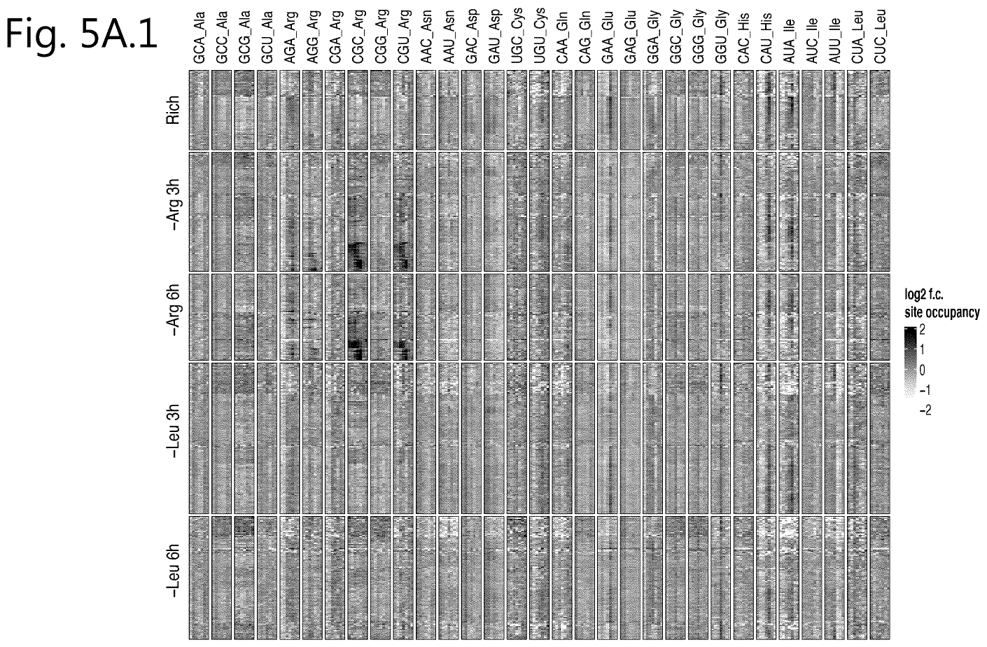

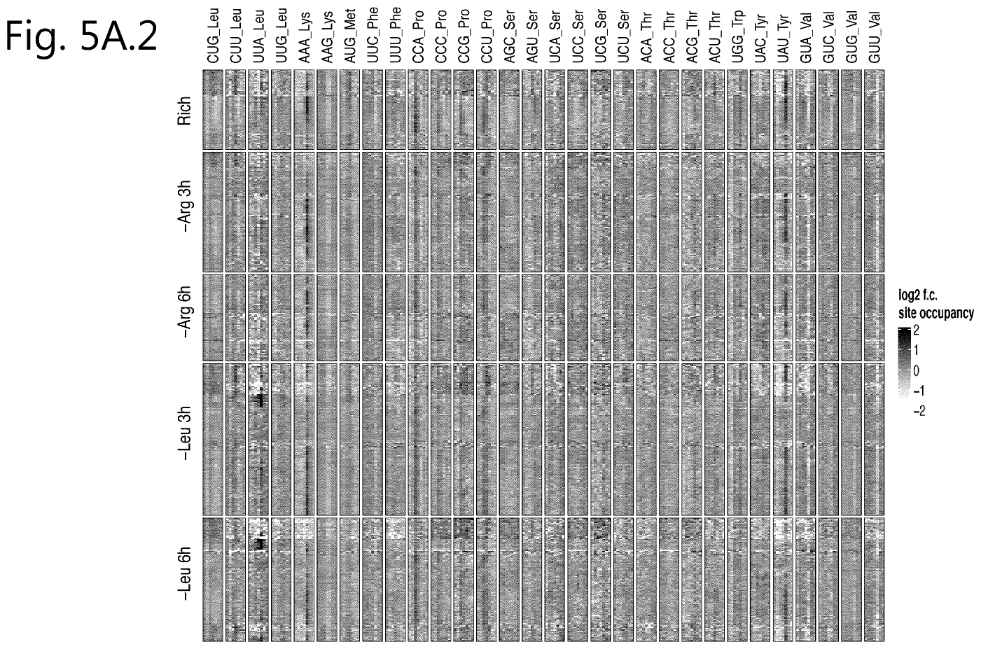

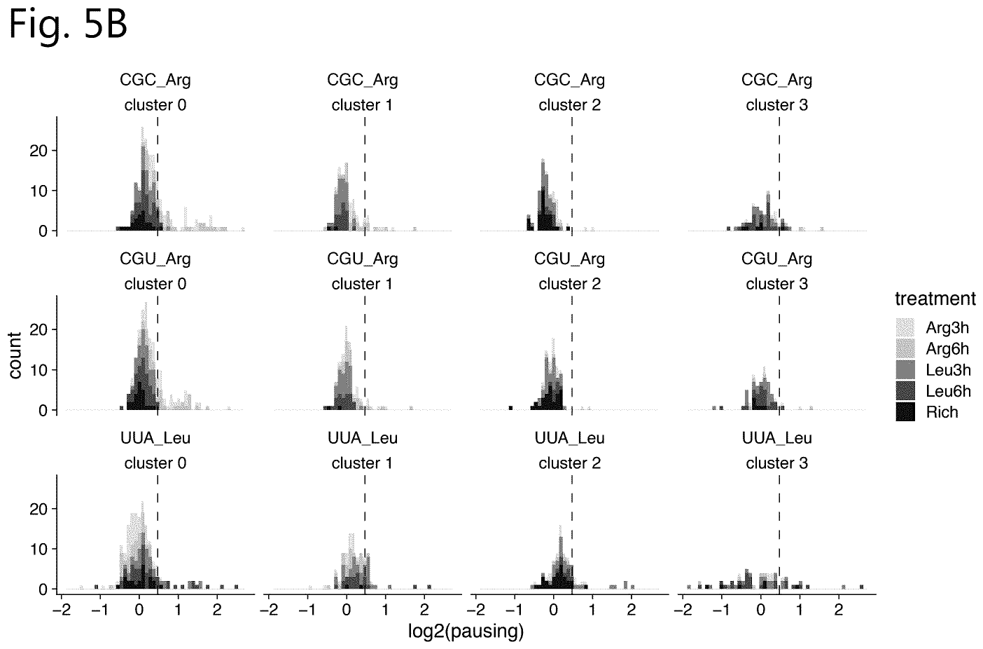

Treatment-specific pausing is also evident in single cells. Reiterating our observations in the pseudobulk analysis, we again see that pausing on arginine and leucine codons is only seen in single cells isolated from the starvation conditions, and only over a subset of codons encoding the removed amino acids (FIG. 2b, FIG. 5a). Furthermore, as the increases in codon occupancies are only apparent in and downstream of the A site, the position in the ribosome footprint where pausing occurs is roughly as expected. This ribosome-site specificity is also apparent in several codons that have been previously associated with ribosome pausing, with, for example, AAA and GAA showing increased occupancies in the A sites (Zinshteyn, B. & Gilbert, W. V. Loss of a conserved tRNA anticodon modification perturbs cellular signaling (2013), PLoS Genet, 9; Nedialkova, D. D. & Leidel, S. A. Optimization of Codon Translation Rates via tRNA Modifications Maintains Proteome Integrity (2015), Cell, 161, 1606-1618), and proline codons in the E sites (Artieri, C. G. & Fraser, H. B. Accounting for biases in riboprofiling data indicates a major role for proline in stalling translation (2014), Genome Res, 24, 2011-2021) (FIG. 5a).

Interestingly, only a subset of the cells from each limitation condition shows a pausing response (69/155 and 53/207 in arginine and leucine limitation, respectively). Clustering cells based on the RPF counts identifies four clusters distinguished by common cell-cycle marker genes with only a subtle effect of the starvation treatments (FIG. 2c, 2f). Based on these clusters, it is apparent that the cell-cycle state has a clear influence on the effect of amino acid limitation on translational pausing. The vast majority of cells that pause under arginine limitation (89.9%) are in either early (cluster 1; 11 cells) or late (cluster 0; 51 cells)S-phase, whereas the cells that respond to leucine limitation are more evenly distributed (FIG. 2d, FIG. 5b).

Ribosome pausing on single genes is also evident in single cells. Examining the RPF density over H3C2, one of the genes that exhibits an increase in CGC pausing under arginine starvation, reveals several pausing hotspots (FIG. 2e,g). The most prominent pausing event on the H3C2 transcript includes two successive CGC codons (FIG. 2e,g), explaining the increased density at this location compared to other identical codons on this transcript. Additionally, these repetitive codons may cause the increase in CGC and CGU occupancy downstream of the A and P sites as seen in FIG. 2b.

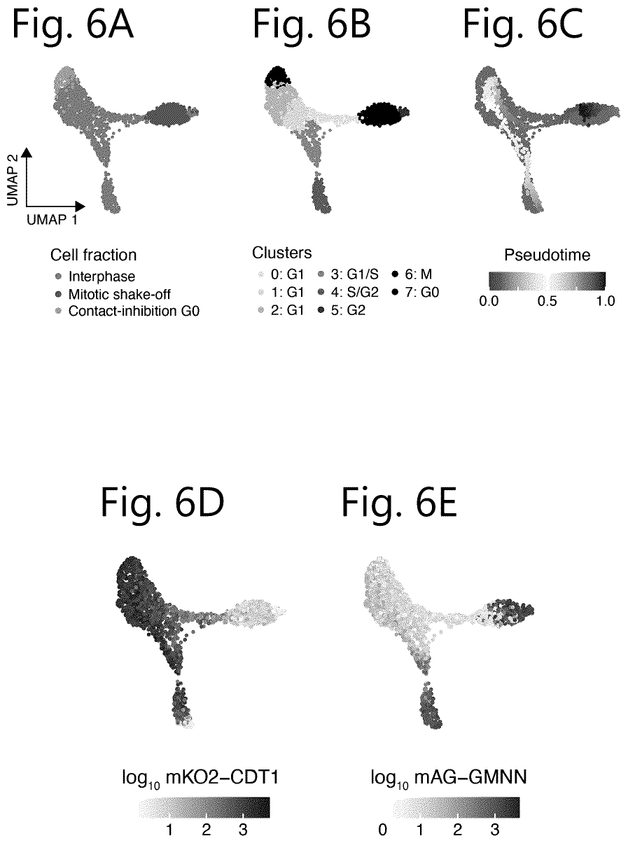

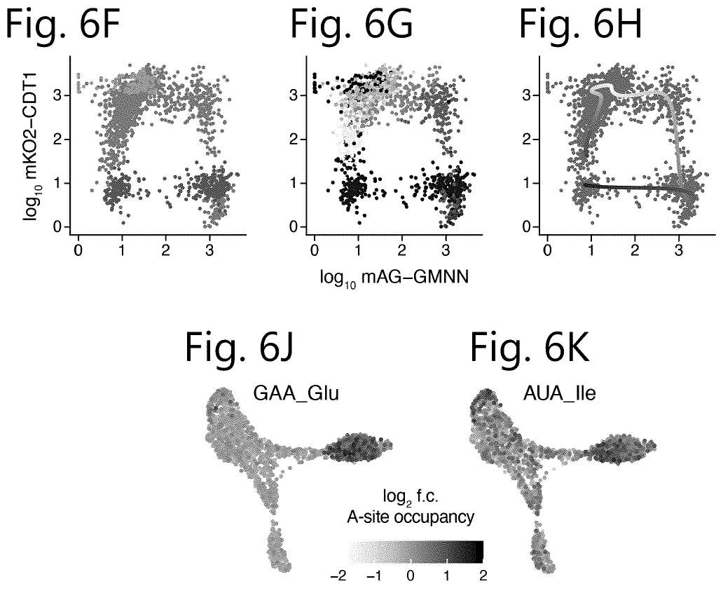

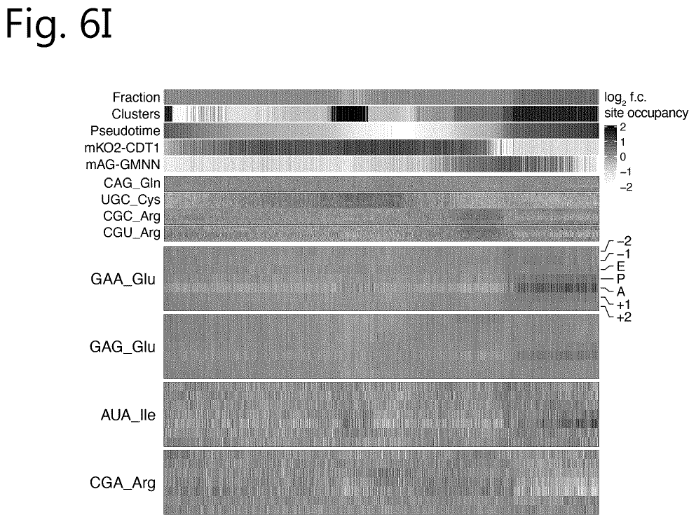

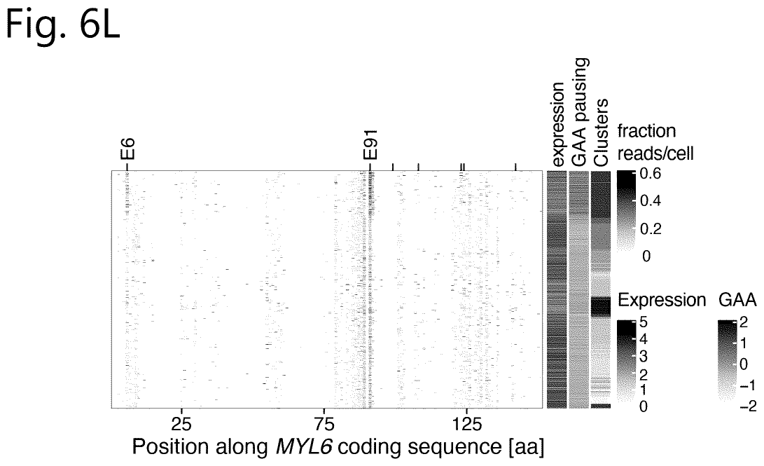

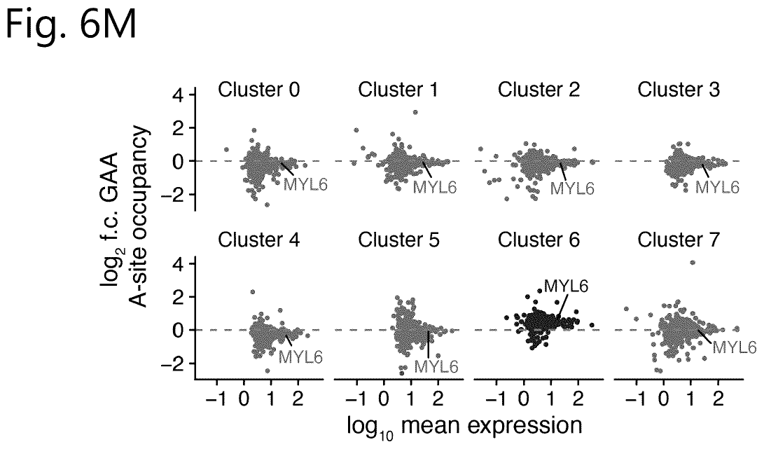

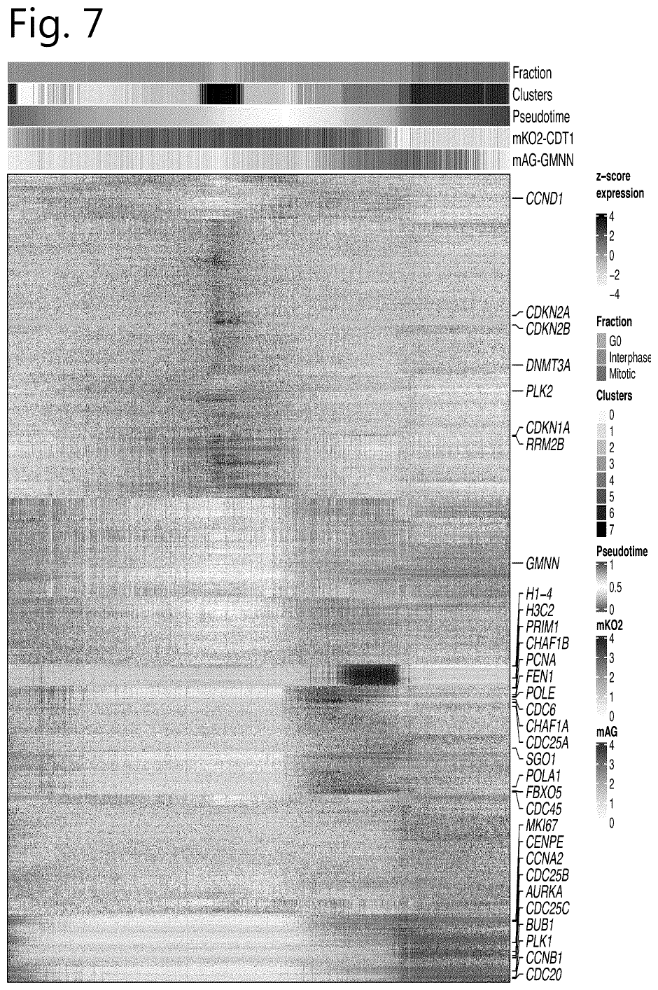

Having seen that the cell cycle state can impact the response to amino acid limitation, we next asked if translational properties changed through the unperturbed cell cycle. Translational regulation has been previously identified as an important cell-cycle control mechanism (Stumpf, C. R., Moreno, M. V., Olshen, A. B., Taylor, B. S. & Ruggero, D. The translational landscape of the mammalian cell cycle. (2013), Mol Cell 52, 574-582, & Tanenbaum, M. E., Stern-Ginossar, N., Weissman, J. S. & Vale, R. D. Regulation of mRNA translation during mitosis. (2015), Elife 4). However, these studies only coarsely resolve the main cell-cycle states and rely on arresting or synchronizing cells with methods that also act on translational machinery (Coldwell, M. J. et al. Phosphorylation of elF4GII and 4E-BP1 in response to nocodazole treatment: a reappraisal of translation initiation during mitosis. (2013), Cell Cycle 12, 3615-3628, & Ly, T., Endo, A. & Lamond, A. I. Proteomic analysis of the response to cell cycle arrests in human myeloid leukemia cells. (2015), Elife 4, & Mettinen, T. P., Kang, J. H., Yang, L. F. & Manalis, S. R. Mammalian cell growth dynamics in mitosis. (2019), Elife 8). We generated scRibo-seq libraries from 1777 single hTERT-RPE1 cells expressing fluorescent ubiquitination-based cell-cycle indicators (FUCCI) (Sakaue-Sawano, A. et al. Visualizing spatiotemporal dynamics of multicellular cell-cycle progression. (2008), Cell 132, 487-498) collected from interphase (1349 cells), contact-inhibition G0 (116 cells), and mitotic shake-off (312 cells) fractions (FIGS. 6a, f). Clustering single cells using the resulting RPF counts identifies eight clusters delineating the main phases of the cell cycle (FIG. 6b). The progression and identity of these clusters closely follows those expected based on fluorescence measurements of the FUCCI markers collected during index sorting (FIGS. 6d, e, g). Pseudotime ordering further resolves this progression through the cell cycle, establishing trajectories through the UMAP projection (FIG. 6c) and FUCCI markers (FIG. 6h), and revealing the translation dynamics of 1531 differentially-translated genes (FIG. 7). Additionally, the change in abundance of several canonical cell-cycle markers follows the expected pattern (FIG. 7b), further confirming the cell ordering.

Surprisingly, in addition to this expected fluctuation in the RPF abundance of numerous genes, the frequency of certain codons in the ribosome footprints also varies over the cell cycle.

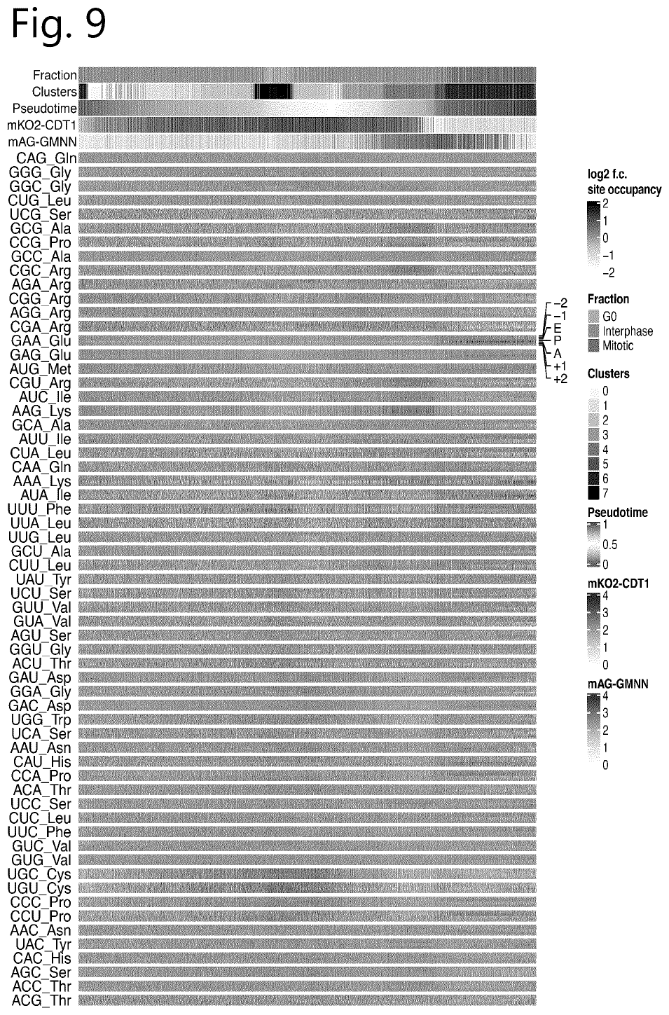

While most codons have constant frequencies of occurrence across ribosome sites and cell-cycle stages (e.g., CAG, FIG. 6i) we identified 14 codons whose frequencies of occurrence in at least one of the ribosome active sites changes throughout the cell cycle (FIG. 9).

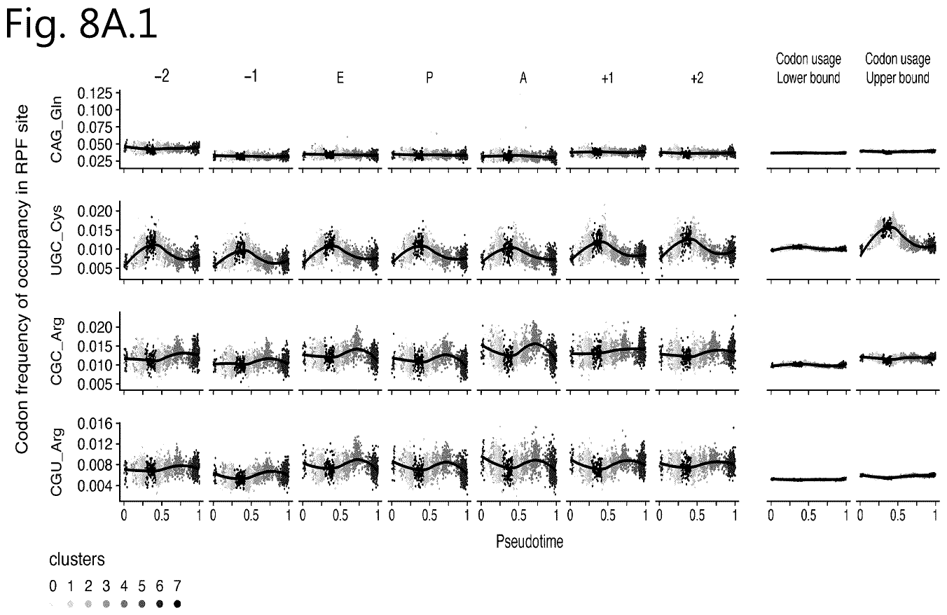

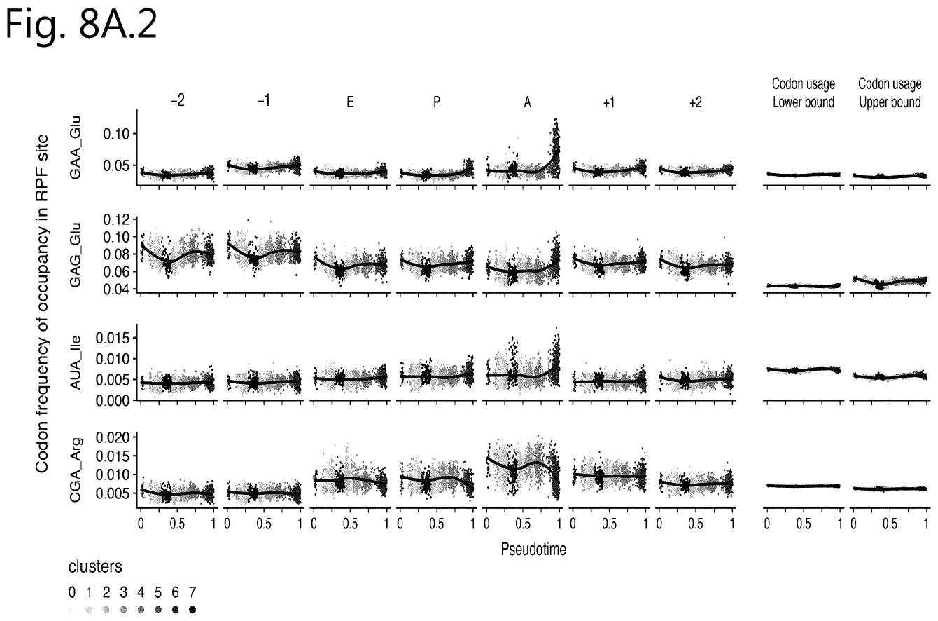

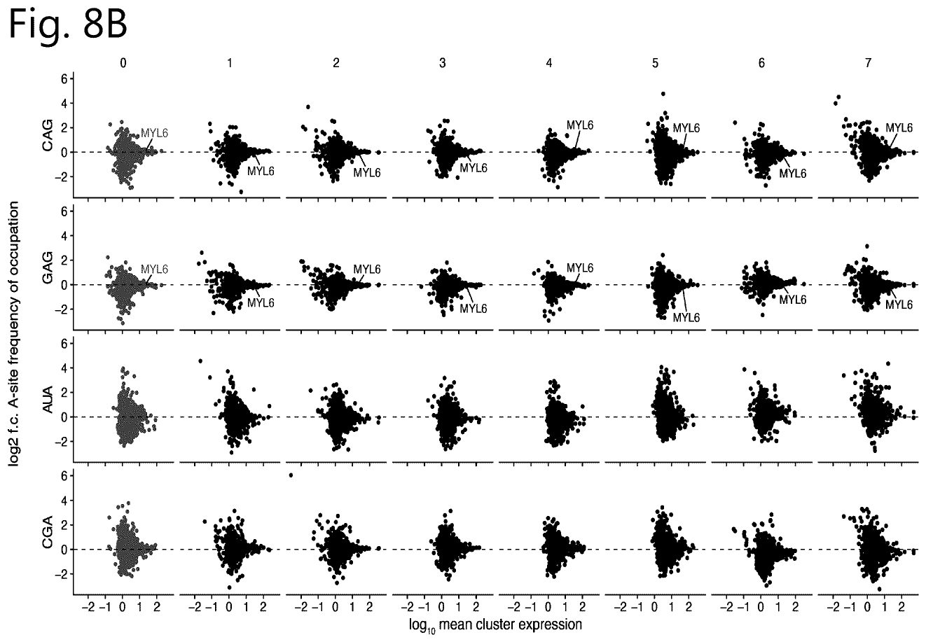

Most of these variable codons display similar changes in occupancy in not only the ribosome E, P, and A-sites, but also in positions immediately up (−1, −2) and downstream (+1, +2). For example, UGC is approximately 1.4 times more likely to occur in all RPF sites in cells in G0 and late G1 [clusters 2 and 7; mean frequency (1.08±0.12) % of RPF sites] than in cells in mitosis [cluster 6; mean frequency (0.78±0.07) % of RPF sites] (FIG. 6i). Interestingly, CGC and CGU, the two codons that show the strongest response to arginine limitation in HEK293T cells (FIG. 2a, b), show these site-agnostic increases in cells in late S phase (cluster 4; FIG. 6i). As this cluster is also marked by the translation of histone genes (FIG. 7), this increase may explain why cells in late S-phase are more susceptible to arginine limitation. However, because these changes are not isolated to specific ribosome active sites and are largely mirrored by changes in codon abundances (FIG. 8a right), they are likely the result of fluctuations in codon usage (Frenkel-Morgenstern, M. et al. Genes adopt non-optimal codon usage to generate cell cycle-dependent oscillations in protein levels. (2012), Mol Syst Biol 8, 572) rather than changes to translational processes.

Conversely, the other codons exhibit site-specific changes in cells undergoing mitosis. Among the codons with variable frequencies of occurrence along the cell cycle are four whose A-site occupancies either increase (e.g., GAA, GAG, and AUA) or decrease (e.g., CGA) in mitotic cells, while the other RPF sites remain constant (mitotic cells: cluster 6; FIG. 6i). Of these, the increase in A-site pausing over GAA is the most pronounced and stage-specific (FIGS. 6i, j), with (6.5±2.1) % of the RPFs from cells in mitosis containing a GAA in the A-site, compared to only (4.0±0.6) % in the other stages. Not all codons follow this same trend, however. For example, cells that are in late mitosis (marked by the sharp decrease in mAG-GMNN fluorescence) have higher AUA pausing than those in early mitosis (FIGS. 6i, k), whereas CGA pausing decreases in mitotic and G0 cells compared to the other stages (FIG. 6i, FIG. 8a).

These changes in A-site pausing are global, affecting the majority of translated genes. Comparing the gene-wise frequency of occurrence of GAA codons in RPF A-sites between each cluster and the background reveals that most genes experience increased GAA pausing during mitosis (FIG. 6m). For example, in mitotic cells (27.7±15.6) % of the RPFs aligning to MYL6 have a GAA in the A-site, with most of these occurring at E6 and E91; in the other stages, only (16.0±11.2) % of the A-sites contain a GAA (FIG. 6l). Averaged across all genes, this is a modest 1.39±0.36 times increase, however, it is widespread, as 37.8% of GAA-containing genes detected across more than three clusters (173/457 detected genes) show a significant increase in A-site GAA pausing in mitotic cells. While not as strong, this same trend is also observed for GAG, AUA, and CGA (FIG. 8b), suggesting that these changes to A-site pausing may reflect global changes in translation dynamics during mitosis.

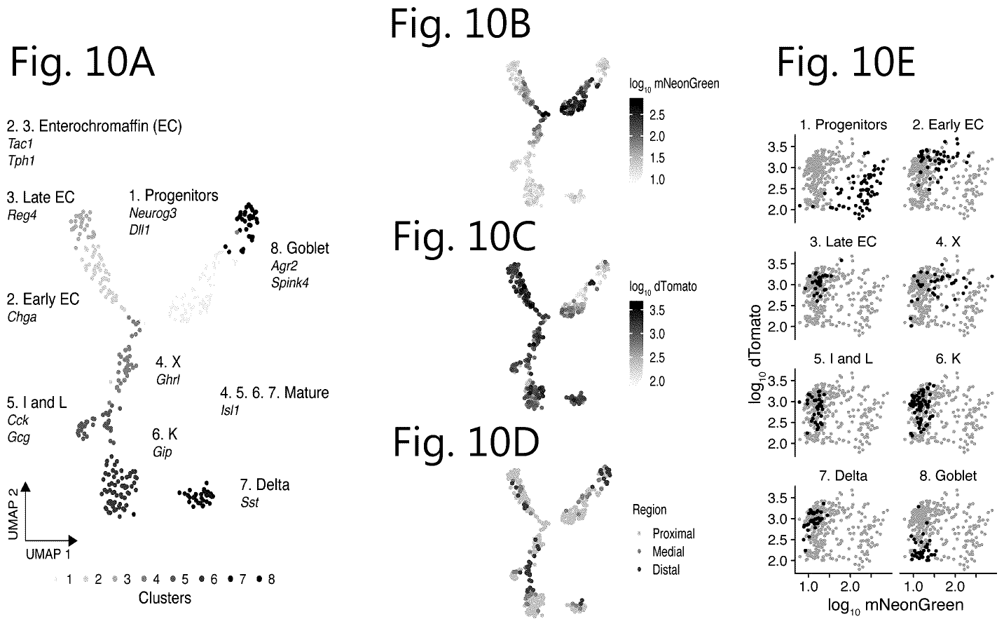

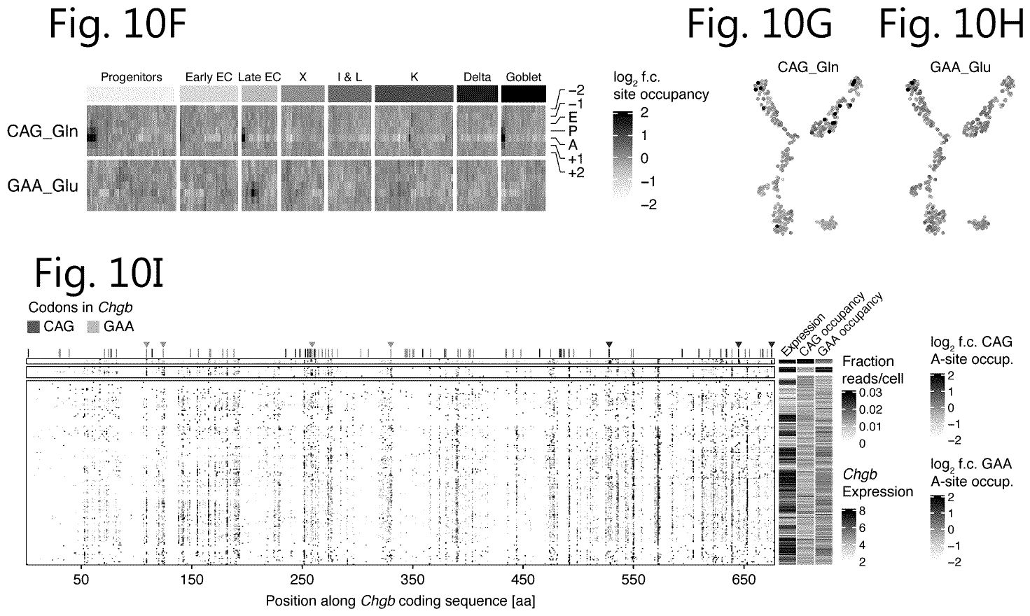

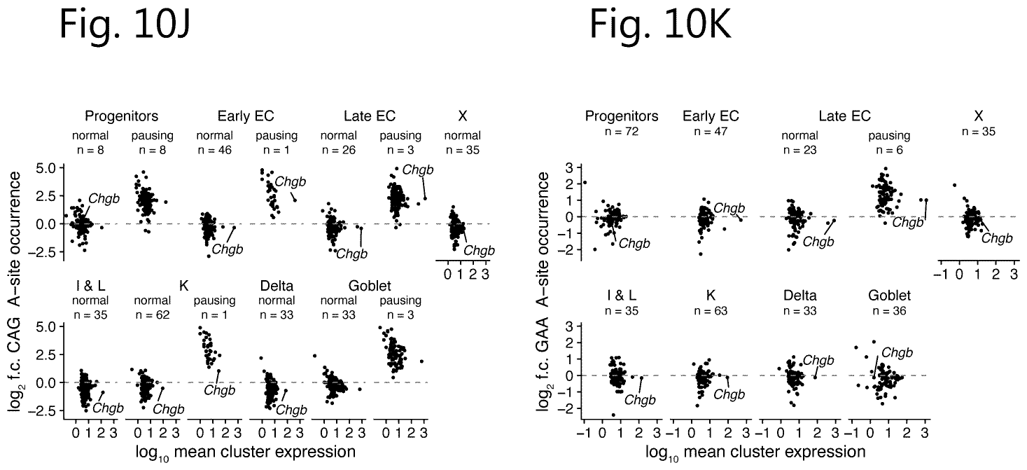

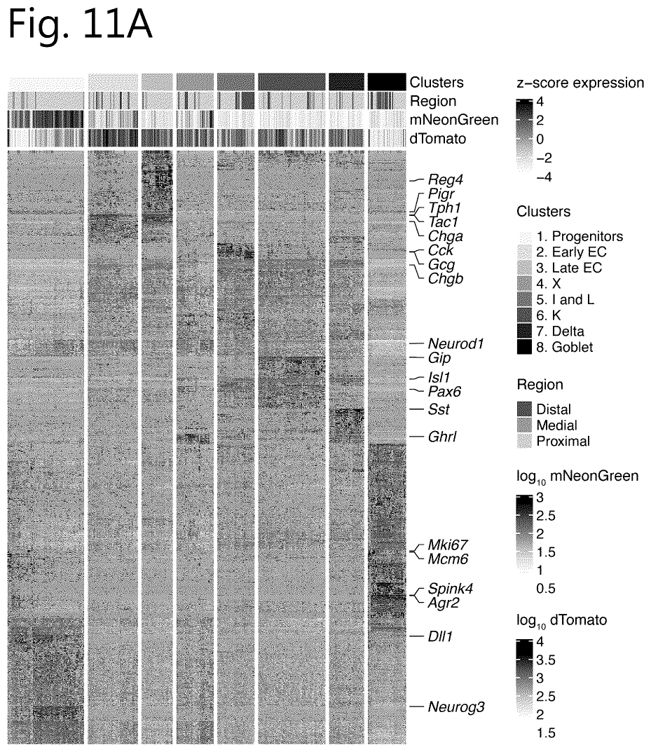

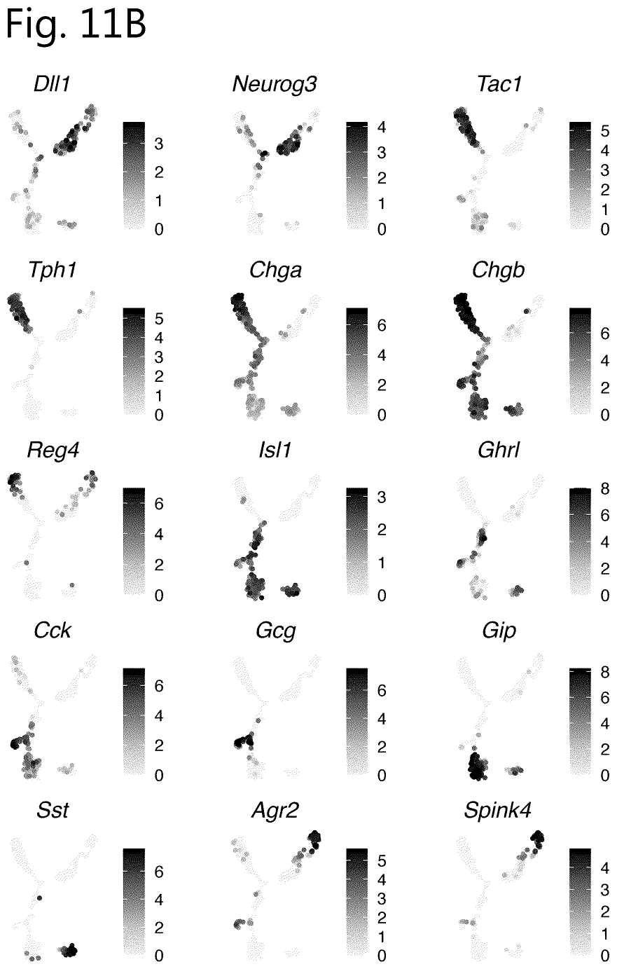

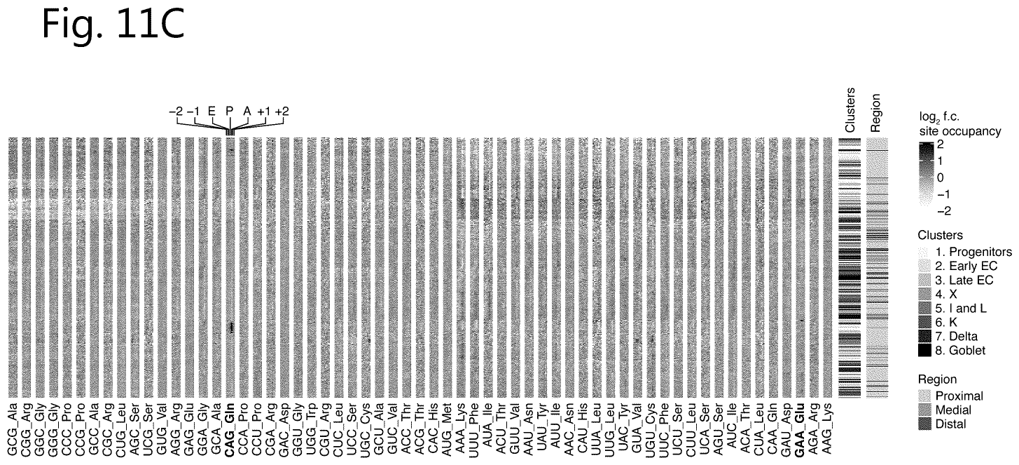

Having demonstrated scRibo-seq on cell lines, we next generated ribosome profiles on primary mouse intestinal enteroendocrine (EEC) cells. EEC cells are a rare population in the gastro-intestinal epithelium (<1%) that produce and secrete diverse hormones in response to nutrient stimuli (Gribble, F. M. & Reimann, F. Enteroendocrine Cells: Chemosensors in the Intestinal Epithelium. Annu Rev Physiol. 78, 277-299 (2016)). They are further subclassified based on the hormones they produce, with the seven cell lineages producing different hormones as they mature, resulting in up to twenty different EEC cell types being described (Gehart, H. et al. Identification of Enteroendocrine Regulators by Real-Time Single-Cell Differentiation Mapping. Cell. 176, 1158-1173 e1116, (2019) & Haber, A. L. et al. A single-cell survey of the small intestinal epithelium. Nature 551, 333-339, (2017)). Their scarcity, diversity, and plasticity make primary EEC cells inaccessible to existing ribosomal profiling methods, making it challenging to study post-transcriptional and translational regulation of their behaviours. We generated ribosomal profiles from 350 single mouse EEC cells expressing a bi-fluorescent Neurog3 reporter (Gehart, H. et al. Identification of Enteroendocrine Regulators by Real-Time Single-Cell Differentiation Mapping. Cell. 176, 1158-1173 e1116, (2019)) isolated from intestinal crypts (FIGS. 10 and 11). Clustering cells based on the RPF counts per CDS identifies 8 clusters representing the main EEC cell types in the crypts that are delineated by the translation of established hormone marker genes (FIGS. 10a, 11a,b). Among the cells are two minority subpopulations that show genome-wide ribosome pausing over CAG-glutamine (n=16 cells) and GAA-glutamic acid (n=6) codons (FIGS. 10f-k, 11c). Interestingly, the GAA-pausing population is only present in the late enterochromaffin cluster (6/29 cells), whereas the CAG-pausing cells were distributed between the cell clusters (GAA: p=1.9×10-7, CAG: p=0.014; Fisher's Exact Test). Together, these results establish that scRibo-seq is directly applicable to complex primary samples, enabling the measurement of translational dynamics in rare cell populations.

scRibo-seq measures translation at the single-cell level, filling a crucial gap in existing capabilities for single-cell genomics. Together, our results demonstrate that scRibo-seq provides a marker- and transgene-free method for ribosomal profiling with the sensitivity and resolution to measure ribosome behaviour down to individual codons on specific transcripts in populations of single cells. Compared to the recently described Ribo-STAMP (Brannan, K. C., I. A.; Yee, B. A.; Marina, R. J.; Lorenz, D. A.; Dong, K. D.; Madrigal, A. A.; Yeo, G. W. Robust single-cell discovery of RNA targets of RNA binding proteins and ribosomes. Nature Methods, (2021)), which uses APOBEC-mediated RNA editing to identify transcripts that have been associated with ribosomes, scRibo-seq provides single-codon resolution and does not require the exogenous expression of a fusion protein. These unique capabilities enabled us to provide a detailed look at translation during the mammalian cell cycle, finding evidence to support widespread changes to translational regulation during mitosis. We anticipate that this method will see broad application, particularly in highly dynamic systems such as development, where rare and short-lived populations are impossible to measure with existing techniques.

There are benefits associated with generating ribosome footprints with MNase, including better preservation of monosome integrity and direct applicability to a wider range of species and tissue types (Darnell, A. M et al, Mol Cell 71, 229-243 e211, 2018; Reid, D. W. et al, Methods 91, 69-74, 2015; Gerashchenko, M. V. & Gladyshev, V. N. Nucleic Acids Res 45, e6, 2017). The method of the invention is however not limited to the use of MNase. Ribonuclease I (RNase I) has a low sequence bias and is thus able to generate ribosome footprints with a high positional accuracy and can further distinguish different ribosome elongation states (Wu, C. C. et al, Mol Cell 73, 959-970 e955, 2019).

To demonstrate that different nucleases can be used instead of MNase in scRibo-seq, we performed a titration of both RNase I and MNase on low-input bulk samples containing approximately 50 RPE-1 cells, scaling up the reaction volumes 50× so that the concentrations match those used when assaying single cells.

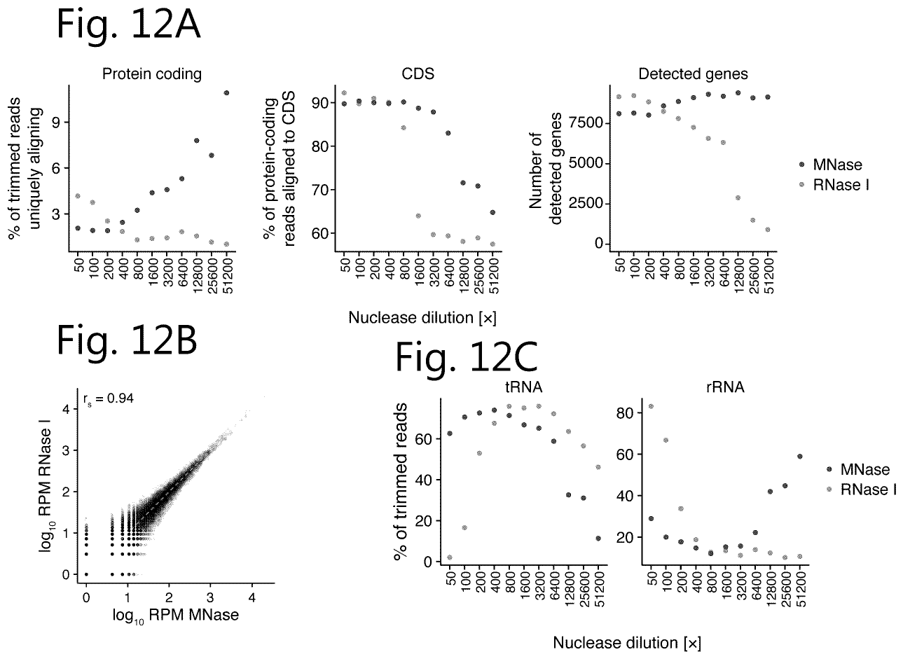

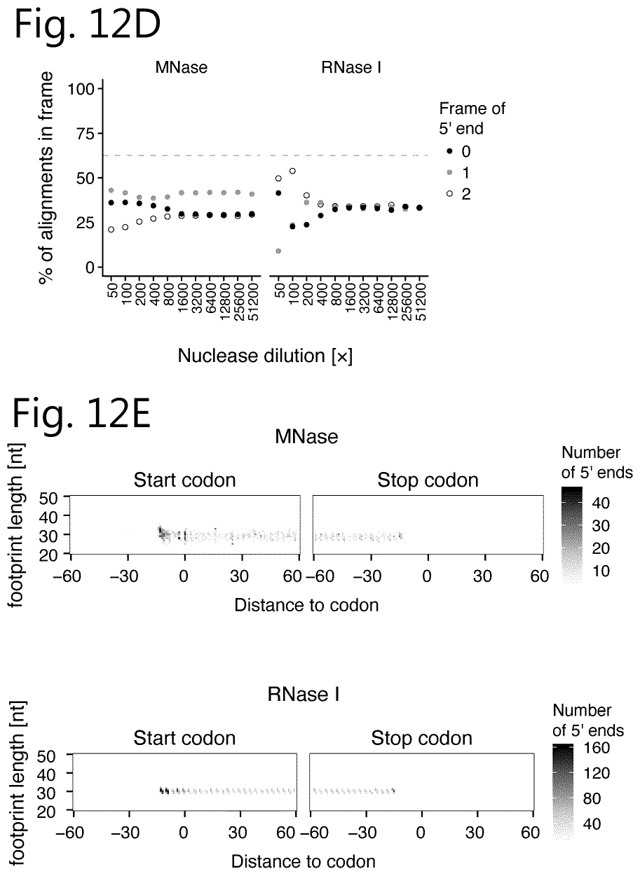

As expected, some of the libraries produced using RNase I have similar performance metrics to those made with MNase. For example, at high concentrations of both nucleases, libraries have similar proportions of reads stemming from protein-coding genes with the majority of those aligning to coding sequences (FIG. 12a). Additionally, the library complexities are also comparable between nucleases, detecting a similar number of genes (FIG. 12a). Finally, the read-depth normalized counts per gene are also highly correlated (Spearman correlation coefficient 0.94, FIG. 12b) between the different nucleases.

In addition to these strong similarities, the different properties of the nucleases are also apparent. First, there is a difference in proportion of reads originating from transfer RNA (tRNA) and ribosomal RNA (rRNA) between nucleases (FIG. 12c). In general, at the high concentrations of both enzymes, libraries produced with RNase I have a higher proportion of rRNA and those produced with MNase have a higher proportion of tRNA. Second, while the proportion of ribosome footprints aligning in each frame along the coding sequence is relatively consistent across different dilutions of MNase (FIG. 12d, left), it is much more variable across concentrations of RNase I (FIG. 12d, right); this is especially prominent in the decrease in the fraction of in-frame reads (frame 0; black) seen between the 1/50× and 1/100× dilutions of RNase I.

The increased positional resolution of RNase I over MNase is also clear. Looking at how the reads map along two metagene regions centered around the start and stop codons (FIG. 11e), we can see that MNase has a broader distribution of read lengths and only a modest 3-nucleotide periodicity in how the reads map. In contrast, the library generated using RNase I is characterized by a sharper distribution of read lengths and a strong 3-nucleotide periodicity in how the reads map.

Together, these results demonstrate that RNase I may be used instead of MNase for single-cell ribosomal profiling. Existing literature has demonstrated that different ribonucleases or combinations of ribonucleases may be interchangeably used to generate ribosome footprints for bulk ribosome profiling (Gerashchenko, M. V. & Gladyshev, V. N, supra), depending on the experimental needs. Our positive results of replacing MNase with RNase I thus underscores that other ribonucleases including RNase A, RNase T1, and ribonuclease combinations may also be used for single-cell ribosomal profiling.

To further demonstrate that the method of the invention (scRibo-seq) produces data with similar characteristics to standard ribosomal profiling methods, we compared several quality-control metrics for the three scRibo-seq datasets to those from four different papers that perform conventional ribosomal profiling. These papers include: 1) a detailed description of the standard RNase-I based ribosome profiling method using HEK293 cells as a demonstration (Ingolia, N. T., et al, Nat Protoc 7, 1534-1550, 2012), 2) a study of smORFs in human cell lines including HEK293T cells that uses RNase I for footprinting (Martinez, T. F. et al. Nat Chem Biol 16, 458-468, 2020), 3) a study of starvation-induced ribosomal pausing in HEK293T cells that uses MNase for footprinting (Darnell A. M., supra), and 4) a study of translation regulation during mitosis in RPE-1 cells that uses RNase I for footprinting (Tanenbaum, M. E. et al, Elife 4, eLife.07957, 2015). These studies, performed by groups with proven experience in ribosomal profiling, capture the data characteristics of standard ribosomal profiling techniques.

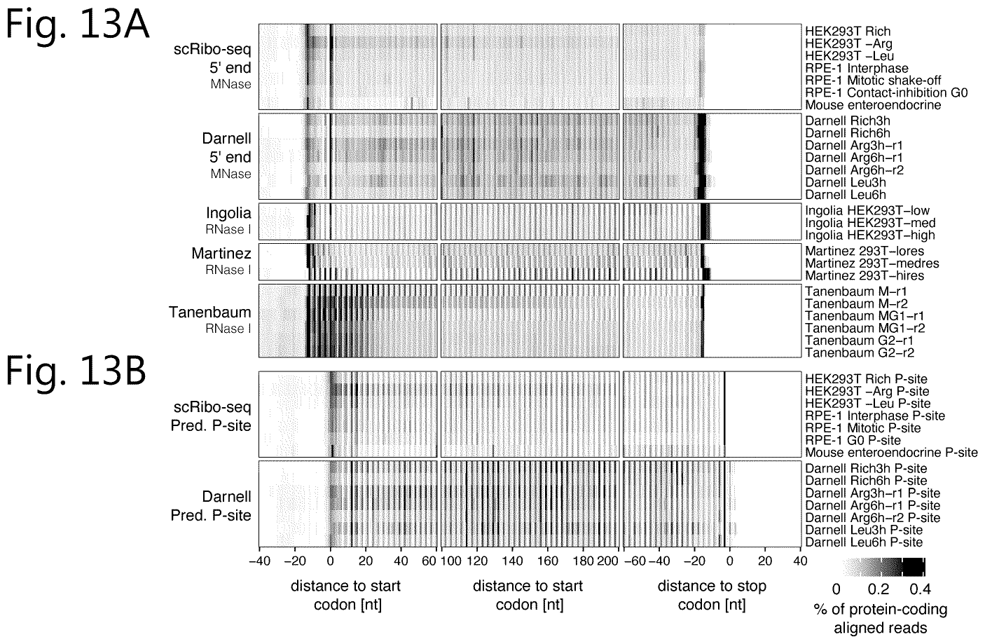

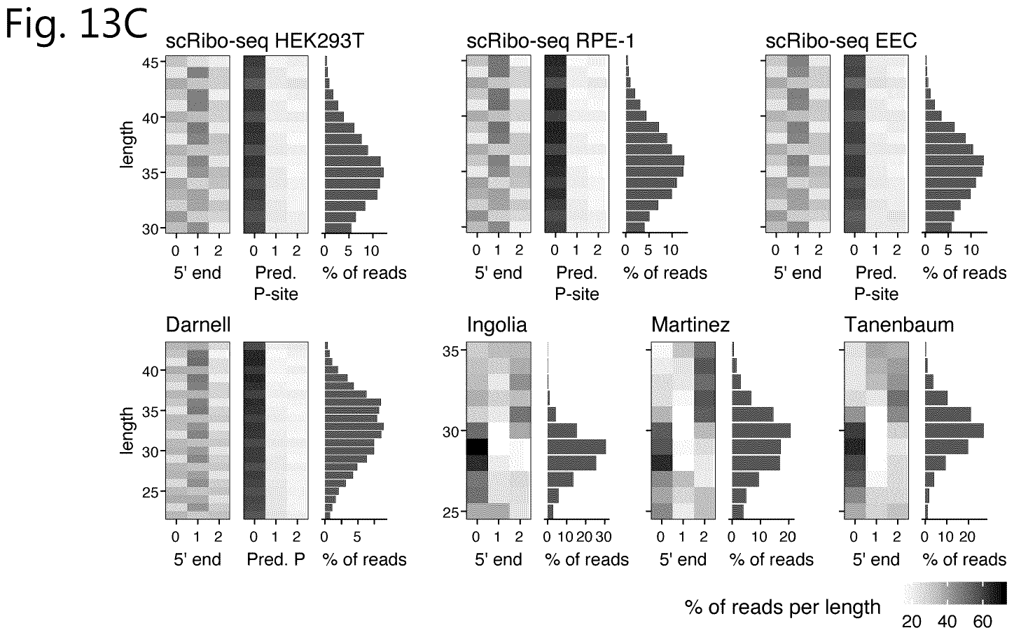

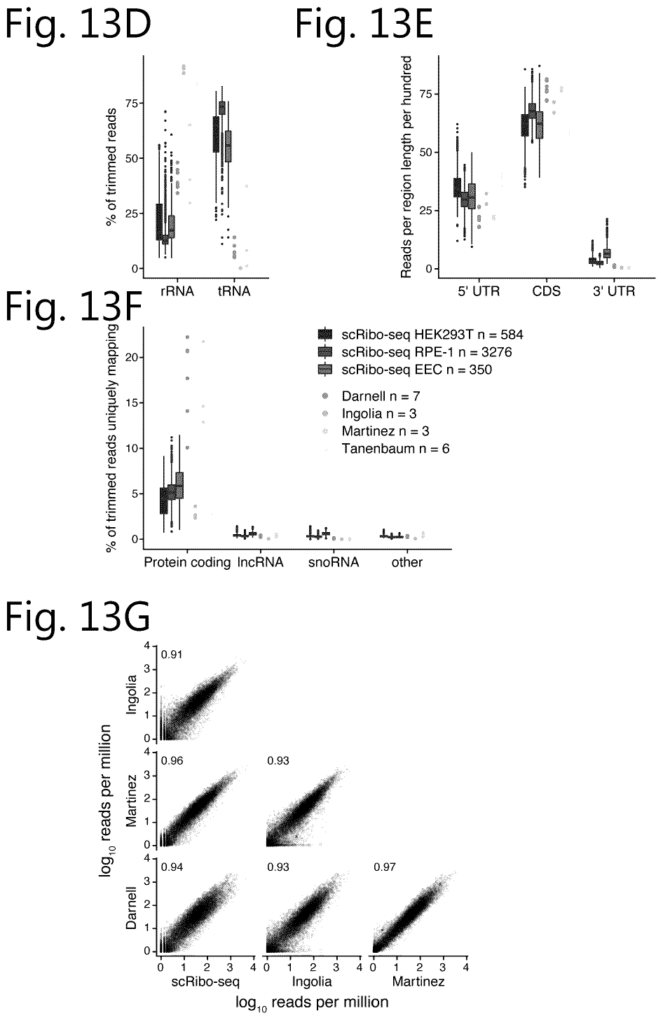

In general, scRibo-seq produces ribosomal profiling libraries with quality metrics that are similar to conventional methods (FIG. 13).

First, the read coverage across the gene body is very similar between all methods (FIG. 13a-b, d-f), with ribosome footprints predominantly mapping to coding sequences. The number of 5′ ends of the fragments sharply increase ˜15 nucleotides upstream of the start codon and decrease ˜18 nucleotides upstream of the stop codon (FIG. 13a-b, left and right panels). There is additionally an increased local density over both the start and stop codons compared to the coding sequence (FIGS. 13a-b).

Second, quantifying these mapping frequencies genome-wide reveals that the distribution of reads between common contaminants, different biotypes, and across the untranslated regions (UTR) and coding sequences (CDS) of protein-coding genes are all similar to those from conventional ribosome profiling methods (Darnell A M, supra; Ingolia N T, supra; Martinez T F, supra; Tanenbaum ME, supra) (FIGS. 13d-f). The largest differences between techniques are in the contribution of common contaminants, where scRibo-seq libraries have a higher fraction of tRNA reads and a lower fraction of rRNA reads (FIG. 13d). In spite of these differences, however, the final proportion of scRibo-seq reads that uniquely align to protein-coding sequences is only surpassed by conventional methods that incorporate a ribosomal depletion step (the Darnell and Martinez datasets; FIG. 13e).

Third, the patterns associated with the MNase digestion are consistent between methods that use this nuclease to generate ribosome footprints. In this comparison, all the scRibo-seq libraries and those generated by Darnell et al (supra) use MNase to produce ribosome footprints. Our observations of a broad distribution of footprint lengths and a complex association between fragment length and the predominant frame of the 5′ end in the scRibo-seq libraries (FIG. 13c, top row) are also visible in the Darnell libraries (FIG. 13c, bottom left).

Finally, the ribosome footprint counts per gene between the methods that profile HEK293T cells are also highly correlated (FIG. 13g). The mean spearman correlation coefficient for these comparisons is 0.94±0.02.

Together these comparisons demonstrate that scRibo-seq produces ribosome profiling libraries with similar performance benchmarks to those produced using traditional high-input methods.

Methods

Cell culture and dissociation. HEK293T cells were obtained from the Medema lab and were cultured in DMEM (Gibco) supplemented with 10% FBS (Gibco), 1×GlutaMAX (Gibco), and 1×Pen-Strep (Gibco) at 37° C. and 5% CO2. For amino acid limitation experiments, HEK293T cells were cultured to ˜70% confluency in “rich” medium based on powdered DMEM medium for SILAC (ThermoFisher Scientific) that was supplemented with 10% dialyzed FBS (ThermoFisher Scientific), 105 mg/L L-leucine (Sigma Aldrich), 84 mg/L L-arginine HCl (Sigma Aldrich), and 146 mg/L L-lysine HCl (Sigma Aldrich). Three and six hours before sorting, cells were washed once with phosphate buffered saline (PBS) and resuspended in medium that did not contain either arginine or lysine. Before sorting, cells were mechanically dissociated to a single-cell suspension by pipetting up and down. DAPI (ThermoFisher Scientific) was added to cultures as a viability stain, and only viable cells were sorted.

RPE-1 hTERT FUCCI cells were obtained from the Medema lab and were cultured in DMEM supplemented with 10% FBS (Gibco), 1× GlutaMAX (Gibco) and 1×Pen-Strep (Gibco) at 37° C. with 5% CO2. For the RPE-1 cell-cycle experiments we used previously characterized RPE-1 hTERT FUCCI cells (Shaltiel, I. A. et al. Distinct phosphatases antagonize the p53 response in different phases of the cell cycle. (2014), Proc Natl Acad Sci USA 111, 7313-7318), and generated three fractions: interphase, mitotic shake-off, and G0-arrested. For the interphase fraction, 7.5×10⁴ cells were plated in a MW-6 and collected by trypsinization (TrypLE, Gibco) 36 hours later. For the mitotic fraction, 3×10 6 cells were plated in a 145 mm dish and were harvested 36 hours later by gently tapping the culture dish and collecting the media (otherwise known as a mitotic shake-off). Finally, for the G0-arrested fraction, 1×10⁵ cells were plated in a MW-24 and collected 72 hours later by trypsinization. DAPI (ThermoFisher Scientific) was added to cultures as a viability stain, and only viable cells were sorted.

Mouse enteroendocrine cells were isolated from the intestines of Neurog3 Chrono mice, closely following the methods outlined by Gehart et al. (Gehart, H. et al. Identification of Enteroendocrine Regulators by Real-Time Single-Cell Differentiation Mapping. Cell 176, 1158-1173 e1116, (2019)). Briefly, mouse small intestines were harvested, cleaned, flushed with PBS0, and separated into proximal, medial, and distal sections. Pieces were cut open and villi were scraped off with a glass cover slip and discarded. Tissue pieces were then washed in cold PBS0 before transferring to PBS0 with 2 mM EDTA (Gibco), incubated at 4° C. for 30 minutes on a roller, and then vigorously shaken. Detached crypts were pelleted, resuspended in warm TrypLE Select (Gibco), and mechanically disrupted by pipetting to generate single-cell suspensions. Single-cell suspensions were washed 2× in Advanced DMEM/F12 (Gibco), strained with a 20-μm mesh, and resuspended in Advanced DMEM/F12 containing 4 mM EDTA and 1 μg/mL DAPI for sorting.

Mice. All mouse experiments were conducted under a project license granted by the Dier Experiment Commissie/Animal Experimentation Committee (DEC) or Central Committee Animal Experimentation (CCD) of the Dutch government and approved by the Hubrecht Institute Animal Welfare Body (IvD). The Neurog3 Chrono allele was maintained on a mixed Mus musculus C57BL/6 background. Animals used in the experiments were aged between 8-22 weeks. Both males and females were used for the experiments. Mice were housed in open housing with 14:10 h light:dark cycle at 24° C. and 45-70% relative humidity with food and water ad libitum. The intestines from two individuals were pooled together during cell dissociation; randomization and blinding were not performed.

FACS. Following dissociation, HEK293T and RPE-1 cells were washed once in 1× PBS0, resuspended in PBS0 with 0.1% bovine serum albumen (BSA; ThermoFisher) and 1 μg/mL DAPI, and passed through a 20-μm mesh. Single cells were index sorted using a BD FACS Influx with the following settings: sort objective single cells, a drop envelope of 1.0 drop, a phase mask of 10/16, extra coincidence bits of maximum 16, drop frequency of 38 kHz, a nozzle of 100 μM with 18 PSI and a flowrate of approximately 100 events per second, which results in a minimum sorting time of approximately 5 minutes per plate.

Doublets, debris, and dead cells were excluded by gating forward and side scatter in combination with the DAPI channel. For the hTERT RPE-1 FUCCI cells, the measurements in the mAG and mKO2 channels were used in combination with the cell preparation treatments to enrich G0 and mitotic populations. For the mouse intestinal enteroendocrine cells, the measurements of dTomato and mNeonGreen were used to select enteroendocrine cells expressing the Neurog3 Chrono reporter and DAPI was used to exclude dead cells. Fluorescence intensities from all channels were stored as index data.

Library construction. Library construction progressed through three general steps (FIG. 1a): cell lysis and ribosome footprint generation, small-RNA library preparation, and pooling and purification. Reagents were dispensed to microwell plates using either the Nanodrop II (Innovadyne Technoligies Inc.) or the Mosquito (TTP Labtech). Plates were spun at 2000×g after each liquid transfer step.

Cell lysis and footprint digestion. Single cells were sorted using a BD FACS Aria into 384-well hardshell plates (BioRad) that were pre-filled with 5 μL of light mineral oil (Sigma Aldrich) and 50 nL of lysis buffer [22 mM Tris-HCl pH 7.5, 16.5 mM MgCl₂, 5.5 mM CaCl₂), 165 mM NaCl, 1.1% Triton X-100, 2.2 U/μL RNaseIN Plus (Promega), 0.11 mg/mL Cycloheximide (Sigma Aldrich)]. After sorting, plates were spun down at 2000×g for 2 minutes and kept on wet ice until all plates were ready for further processing. Next, 50 nL of Micrococcal Nuclease (MNase, 10500 U/mL, New England Biolabs) was added to each well, and plates were incubated at 37° C. for 30 minutes. In order to stop digestion, 50 nL of stop mix [0.0186 U/μL Thermolabile Proteinase K (New England Biolabs), 62 mM EGTA (Sigma Aldrich), 16.5 mM EDTA (Ambion), and 697.5 mM guanidium thiocyanite (GuSCN, Sigma Aldrich)] was added to each well, and plates were incubated at 37° C. for 30 minutes then 55° C. for 10 minute and held at 4° C.

Small RNA library preparation. After ribosome footprint digestion, libraries were constructed using a one-pot small-RNA library preparation protocol that incorporated end repair, two RNA ligations, cDNA synthesis, and an indexing PCR. First, 50 nL of end-repair mix [4.1×of 10× T4 RNA Ligase Buffer (New England Biolabs), 16.4 mM MgCl₂, 4.1 mM uridine triphosphate (New England Biolabs), 1.37 U/μL T4 Polynucleotide Kinase (New England Biolabs), and 0.82 U/μL RNaseIN Plus] was added to each well, and plates were incubated at 37° C. for 1 hour and held at 4° C. Next, 264 nL of 3′ ligation brew [1× T4 RNA Ligase Buffer (New England Biolabs), 1 μM pre-adenylated 3′ adapter (Integrated DNA Technologies), 35.5% PEG-8000 (New England Biolabs), 0.1% Tween-20 (Sigma Aldrich), 1 U/μL RNaseIN Plus, and 21.3 U/μL T4 RNA Ligase 2 Truncated KQ (New England Biolabs)] was added to each well and plates were incubated at 4° C. for 18 hours. The cDNA synthesis primer was then pre-annealed to the 3′ ligation products by adding 50 nL of the RT primer mix [5.2 μM RT primer (Integrated DNA Technologies), 13.5 μM adenosine triphosphate (ATP, New England Biolabs), and 1% Tween-20] to each well, heating to 65° C. for 1 minute, 37° C. for 2 minutes, 25° C. for 2 minutes, and holding at 4° C. Five-prime adapters were then ligated by adding 156 nL of 5′ ligation brew [1×T4 RNA Ligase Buffer, 30.75% PEG-8000, 0.1% Tween-20, 0.5 μM 5′ adapter (Integrated DNA Technologies), 1.25 U/μL T4 RNA Ligase 1 (Ambion)] and incubating at 37° C. for 2 hours and holding at 4° C. Complementary DNA synthesis was then performed by adding 771 nL of reverse transcription brew [1.88× 5× RT Buffer (ThermoFisher Scientific), 1.25 mM dNTPs (Promega), 0.1875% Tween-20, 1.875 U/μL RNaseIN Plus, and 9.375 U/μL Maxima H Minus Reverse Transcriptase (ThermoFisher Scientific)] to each well, and heating at 50° C. for 1 hour, then 85° C. for 5 minutes and holding at 4° C. Finally, single-cell libraries were indexed during PCR by first transferring 150 nL of 20 μM unique forward index primers (Integrated DNA Technologies) and 3.2 μL of PCR brew [1.5× Q5 Hot Start High-Fidelity 2× Master Mix (New England Biolabs), 0.15% Tween-20, and 0.94 μM reverse index primer (tIntegrated DNA Technologies)] to each well. Plates were then incubated at 98° C. for 30 s followed by 10 cycles of 98° C. for 15 s, 65° C. 30 s, 72° C. for 30 s, and then a final incubation at 72° C. for 5 min and holding at 4° C. Plates were then frozen at −20° C. until pooling.

Pooling and purification. After library construction the plates were pooled and purified. The contents of each plate were first collected in VBLOK200 reservoirs (Click Bio) by centrifuging at 2000×g for 2 min. The aqueous phase (˜1.9 mL per plate) was separated from the light mineral oil by centrifugation, and concentrated to approximately 500 μL using n-butanol (Sigma Aldrich) and diethyl ether (Sigma Aldrich). Product was then cleaned up using AMPure XP beads (Beckman Coulter) that had been diluted 5× in bead binding buffer [20% PEG-8000 (Sigma Aldrich) 2.5 M Sodium Chloride (Sigma Aldrich)]; diluted beads were added to the sample at a 2.1:1 ratio, and the final product was resuspended in 50 μL of low TE buffer [LoTET, 3 mM Tris-HCl pH 8.0 (Ambion), 0.2 mM EDTA pH 8.0 (Gibco), 0.1% Tween-20]. Half of each of the cleaned-up library pools was then run on a 10 cm 7% polyacrylamide gel at 200 V for ˜6 h, and the ˜10 base-pair region from 175-185, corresponding to an insert size of ˜30-40 nt was excised. The band was then crushed and soaked overnight at 4° C. in elution buffer [5:1 LoTET:7.5 M ammonium acetate (Sigma Aldrich)]. Eluate was finally precipitated in ethanol.

Sequencing. Libraries were sequenced using v2.5 chemistry on a NexSeq 500 (Illumina) with 75 cycles for read 1, 6 cycles for the i7 index read (plate index), and 10 cycles for the i5 index read (cell index).

Data Analysis-Reference genomes. The human reference genome and annotations were obtained from Gencode Release 34 (GRCh38.p13) and mouse release 24 (GRCm38.p6). The reference genome was prepared for alignment by masking all tRNA genes and pseudogenes and including unique pre-tRNAs genes as artificial chromosomes. tRNA genes and pseudogenes were identified using tRNAscan-SE (version 2.0.5) using the eukaryotic model (−HQ) and the vertebrate mitochondrial model (−M vert −Q). Sequences for ribosomal RNAs were downloaded from NCBI

RefSeq (human: 12S RNR1, 16S RNR2, RNA45SN5, RNA45SN1, RNA45SN4, RNA45SN2, RNA45SN3, RNA5S9, RNA5S1-17; mouse: Rn45s, Rn5s, 12s 16s, and Rn47s). For metagene analyses, a set of canonical transcripts was defined based on the APPRIS annotations, with the longer isoforms being selected in cases of multiple primary isoforms.

Data Analysis-Read processing. Reads were first demultiplexed using bcl2fastq (version 2.20.0.422) with—use-bases-mask Y*,I*,Y* —no-lane-splitting—mask-short-adapter-reads 0—minimum-trimmed-read-length 0. Next, the UMI was extracted from the first 10 bases of read 1 and concatenated to the start of the cell barcode. Adapter sequences were then trimmed from read 1

1. A method for determining a translatome of a cell, comprising the steps of:
i) lysing a single cell;
ii) digesting the RNA with a ribonuclease, thereby generating a ribosome footprint containing RNA molecules that are protected against digestion;
iii) inactivating the ribonuclease and releasing the RNA molecules from the ribosomes;
iv) end repairing the released RNA molecules;
v) constructing an RNA library from the end-repaired RNA molecules;
vi) size selecting part of the prepared RNA library for fragments having an insert size of about 20-40 nucleotides;
vii) sequencing the size selected RNA library; and
viii) determining the translatome of the cell,
wherein preferably the cell is a single cell.

2. A method according to claim 1, wherein the ribonuclease in step ii) is a micrococcal nuclease (MNase).

3. A method according to claim 1, wherein in step iii) the ribonuclease is inactivated by a thermolabile proteinase K and/or the presence of a chelating agent.

4. A method according to claim 3, wherein the chelating agent is at least one of EDTA and EGTA.

5. A method according to claim 1,
wherein step iii) further comprises the presence of a chaotropic agent, wherein the chaotropic agent is preferably guanidium thiocyanite (GuSCN).

6. A method according to claim 1,
wherein in step iv) a polynucleotide kinase (PNK) and a phosphate donor is used to end repair the released RNA molecules.

7. A method according to claim 6, wherein the phosphate donor is not ATP, preferably wherein the phosphate donor is selected from the group consisting of UTP, CTP, GTP, TTP, dATP and dTTP.

8. A method according to claim 1,
wherein the translatome of two or more cells are determined.

9. A method according to claim 8, wherein the method comprises a step of pooling the constructed RNA libraries after step v) and before step vi).

10. A method according to claim 1,
wherein the library preparation step v) comprises the sub-steps of:
a) ligating a first adapter to the 3′-end and a second adapter to the 5′-end of the end-repaired RNA molecules, wherein preferably at least one of the first and second adapter comprises at least one of an UM and a barcode;
b) reverse transcribing the adapter-ligated RNA molecules to obtain cDNA; and
c) amplifying the cDNA with a first and a second primer, wherein preferably at least one of first and second primer comprises a barcode.

11. A method according to claim 10, wherein the barcode in step a) and/or step c) is at least one of a cell barcode, a sample barcode and a plate barcode.

12. A method according to claim 10, wherein sub-step a) of ligating the first and/or second adapter is performed at a temperature below about 10° C., preferably at a temperature of about 4° C., preferably for a time period of at least about 0.5, 1, 2, 4, 6, 8, 10, 12, 14 or 16 hours.

13. A method according to claim 10,
wherein sub-step a) of ligating the first and/or second adapter is performed in a buffer comprising polyethylene glycol (PEG), preferably PEG-8000, wherein the concentration PEG is preferably about 30%-40%, preferably about 30%, 31%, 32%, 33%, 34%, 35%, 36%, 37%, 38%, 39% or 40% or preferably about 15%-25%, preferably about 15%, 16%, 17%, 18%, 19%, 20%, 21%, 22%, 23%, 24% or 25%.

14. A method according to claim 10, further comprising a complexity reduction step, wherein the complexity reduction step is preferably an amplification step d), wherein at least one of the primers comprises a selective nucleotide at the 3′-end for amplification of a subset of nucleotides.

15. A method according to claim 1,
wherein at least one of
the cell is a mammalian cell, preferably a human cell, preferably a human tumor cell or an embryonic cell; and
the method does not comprise an RNA purification step.
